(19) United States
(12) Patent Application Publication
(10) Pub. No.: US 2014/0170723 A1
(43) Pub. Date: Jun. 19, 2014

(54) Sequential Fermentation of Hydrolsate and Solids from a Dilute Acid Hydrolysis of Biomass to Produce Fermentation Products

(71) Applicants: Ian Dobson, London (GB); John Doyle, Southborough, MA (US); Charles Isaac, Carlsbad, IL (US); Andrew MacDonald, Lafayette, LA (US); Katherine Smart, Rempstone (GB)

(72) Inventors: Ian Dobson, London (GB); John Doyle, Southborough, MA (US); Charles Isaac, Carlsbad, IL (US); Andrew MacDonald, Lafayette, LA (US); Katherine Smart, Rempstone (GB)

(21) Appl. No.: 14/105,698

(22) Filed: Dec. 13, 2013

Related U.S. Application Data
(60) Provisional application No. 61/737,558, filed on Dec. 14, 2012.
(60) Provisional application No. 61/737,565, filed on Dec. 14, 2012.

(51) Int. Cl. C12P 7/10

(52) U.S. Cl. CPC C12P 7/10
USPC 435/165

(57) ABSTRACT

A method of producing renewable material comprising (a) converting biologically a hemicellulose-derived material to form a first mixture comprising a first renewable material, and (b) convening a substantial amount of a material comprising cellulose and lignin in the presence of at least a portion of the first mixture to form a second mixture comprising second renewable material.

This application claims the benefit of U.S. Provisional Patent Application Ser. No. 61/737,558 filed on Dec. 14, 2012, and U.S. Provisional Patent Application No. 61/737,565 filed on Dec. 14, 2012, and titled, “Process for the Conversion of Cellulosic Feedstock Materials,” which are incorporated herein by reference in its entirety.

### BACKGROUND OF THE INVENTION

1. Field of the Invention

The present invention relates to the production of fuels or chemicals utilizing lignocellulosic biomass as a feedstock.

2. Background of the Invention

A desire to produce fuels and chemical building blocks from renewable sources, as well as the desire to reduce the introduction of greenhouse gases such as carbon dioxide in the atmosphere by, for example, the combustion of fossil fuels, have led to development of technologies seeking to utilize natural cycles between fixed carbon and liberated carbon dioxide. A fuel produced from material having the carbon atoms contained in, for example, a feedstock such as a lignocellulosic or cellulosic plant material, will return the carbon in the form of carbon dioxide to the pool of carbon dioxide in the atmosphere when combusted thr example, eternal combustion engine of an automobile, truck, locomotive, or aircraft. The growth of new plants of lignocellulosic or cellulosic feedstock materials will use carbon dioxide from that pool thereby completing a cycle of feedstock growth, conversion to fuel, fuel utilization, and re-growth without increasing the amount of carbon dioxide to the atmosphere.

As these technologies advance, various techniques to convert cellulosic or lignocellulosic feedstock materials into fuels have been developed. However, even with these advances, there remains a need and a desire to improve the efficiency of the conversion of renewable carbon sources, such as cellulosic or lignocellulosic feedstock materials to fuels and chemical building blocks.

Processes in accordance with the present invention generally can result in reduced water consumption.

Processes in accordance with the present invention generally can result in converting the maximum amount of liberated sugars from lignocellulosic feedstock into fuels and chemicals thereby increasing the overall yield.

Processes in accordance with the present invention generally can result in requiring a lower dose of a fermentation organism.

Processes in accordance with the present invention generally can result in requiring less fermentation reactor volume thereby reducing the overall capital expenditure of an ethanol production facility.

Other aspects will become readily apparent upon consideration of the figures and ensuing description.

### SUMMARY OF THE INVENTION

Now therefore, what is provided in a first embodiment of the present invention is a method of producing renewable material comprising:

(a) converting biologically a hemicellulose-derived material to form a first mixture comprising a first renewable material; and

(b) converting a material comprising cellulose and lignin in the presence of at least a portion of the first mixture to form a second mixture comprising a second renewable material.

In one embodiment, the step of converting in step (b) comprises saccharification of the material,

In one embodiment, the second renewable material is glucose.

In one embodiment, the method further comprises the step of fermentation of the second renewable material formed in step (b).

In one embodiment, the fermentation and saccharification occur simultaneously.

In one embodiment, the saccharification comprises enzymatic hydrolysis.

In one embodiment, the fermentation is performed by a microorganism.

In one embodiment, the microorganism used in step (b) is selected from one or more of Escherichia, Zymomonas, Saccharomyces, Candida, Pichia, Streptomyces, Bacillus, Schizosaccharomyces, Dekkera, Bretanomyces, Kluyveromyces, Issatchenkia, Hansenula, Pachysolen, Torulaspora, Zygosaccharomyces, Yarrowia, Lactobacillus, and Clostridium.

In one embodiment, the step of converting in step (a) comprises fermentation.

In one embodiment, the hemicellulose-derived material comprises xylose and xylooligomers.

In one embodiment, the fermentation of step (a) is performed by a microorganism.

In one embodiment, the microorganism used in step (a) is selected from one or more of Escherichia, Zymomonas, Saccharomyces, Candida, Pichia, Streptomyces, Bacillus, Schizosaccharomyces, Dekkera, Bretanomyces, Kluyveromyces, Issatchenkia, Hansenula, Pachysolen, Torulaspora, Zygosaccharomyces, Yarrowia, Lactobacillus, and Clostridium.

In one embodiment, the first and second renewable materials are the same.

In one embodiment, the first and second renewable materials are selected from the group consisting of alcohols and organic acids.

In one embodiment, the alcohol is selected from the group consisting of ethanol and butanol.

In one embodiment, the microorganism used in step (a) and step (b) is the same.

In one embodiment, the hemicellulose-derived material and the material comprising cellulose and lignin are obtained from hydrolysis of a lignocellulosic feedstock.

In one embodiment, hydrolysis comprises the step of contacting the feedstock with an acid.

In one embodiment, the hemicellulose-derived material is detoxified prior to converting in step (a).

In one embodiment, hydrolysis is catalyzed by an acid.

In one embodiment, the feedstock is selected from the group consisting of Miscanthus, Erianthus, energy cane, sugar cane, sorghum, Napier grass, and switch grass.

In one embodiment, the first mixture reduces the viscosity of the second mixture.

In one embodiment, step (a) and step (b) occur in the same vessel.

In one embodiment, step (a) and step (b) occur in different vessels.

In one embodiment, the enzyme for the enzymatic hydrolysis is selected from the group comprising cellobiohydrolase I, cellobiohydrolase II, beta-glucosidase, endoglucanase or any combination thereof.

In one embodiment, the second renewable material is a sugar.

In one embodiment, the sugar comprises glucose.

In a second embodiment, the present invention provides a method for producing an alcohol comprising:

(a) fermenting xylose in a mixture comprising a quantity of xylose during a first time to form a first mixture comprising an alcohol and unfermented xylose;

(b) saccharifying a material comprising cellulose and lignin to form glucose in a second mixture comprising at least a portion of the first mixture; and

(c) fermenting in the second mixture the glucose in the second mixture and at least a portion of the unfermented xylose from the first mixture, to form an alcohol.

In one embodiment, the fermenting in step (c) and the saccharifying occur simultaneously.

In one embodiment, the fermenting in step (a) and the fermenting in step (b) are in separate vessels.

In one embodiment, the fermenting in step (a) and the fermenting in step (b) are in the same vessel.

In one embodiment, the alcohol is selected from the group consisting of ethanol and butanol.

In one embodiment, the alcohol is ethanol.

In one embodiment, the alcohol is n-butanol.

In one embodiment, the alcohol is isobutanol.

In a third embodiment, the present invention provides a method of producing renewable material comprising:

(a) hydrolyzing in the presence of water a feedstock comprising lignin, cellulose and hemicellulose to form a liquid portion comprising hemicellulose-derived material and a solids portion comprising cellulose and lignin;

(b) separating at least a substantial amount of the liquid portion from the solids portion to form a liquid fraction and a solids fraction;

(c) converting biologically in the presence of water at least a portion of the hemicellulose-derived material in the liquid fraction to form a first mixture comprising water and a first renewable material;

(d) combining at least a portion of the first mixture with at least a portion of the solids fraction to form a second mixture having improved pumpability relative to the solids fraction; and

(e) converting at least a portion of cellulose in the second mixture to form a second renewable material.

In one embodiment, the solids fraction has a solids content of about 30% to about 35%.

In one embodiment, the solids fraction has a solids content of about, 20%, 21%, 22%, 23%, 24%, 25%, 26%, 27%, 28%, 29%, 30%, 31%, 32%, 33%, 34% or 35%.

In one embodiment, the solids content of the second mixture is about 10% w/v to about 15% w/v, and more specifically, about 12% w/v to about 14% w/v, and even more specifically about 13% w/v.

In one embodiment, the solids content of the second mixture is about 10%, 11%, 12%, 13%, 14% or 15% w/v.

In a fourth embodiment, the present invention provides a method of producing renewable material comprising:

(a) converting biologically at least a portion of a hemicellulose-derived material to form a first mixture comprising a first renewable material; and

(b) converting biologically at least a portion of a cellulose-derived material in the presence of at least a portion of the first mixture and lignin to form a second mixture comprising a second renewable material.

In a fifth embodiment, the present invention provides a process for producing a renewable material comprising:

(a) pretreating a feedstock to provide liquid fraction comprising a hemicellulose-derived material and a solid fraction comprising cellulose and lignin;

(b) separating the liquid fraction from the solid fraction;

(c) converting biologically at least a portion of a hemicellulose-derived material to for a first mixture comprising a first renewable material; and

(d) converting at least a portion of a material comprising cellulose and lignin in the presence of at least a portion of the first mixture to form a second mixture comprising a second renewable material.

In a sixth embodiment, the present invention provides a process for producing a renewable material comprising:

(a) charging a reactor with a first amount of hemicellulose-derived material;

(b) converting biologically at least a portion of the first amount of a hemicellulose-derived material in a the reactor for an initial time period of about 1 hour to about 48 hours to form a mixture comprising a renewable material and residual hemicellulose-derived material;

(c) charging the reactor with a second amount of a hemicellulose-derived material and lignin after the initial time period; and

(d) converting biologically at least a portion of the second amount of hemicellulose-derived material from step (c) and residual hemicellulose-derived material from step (b) to form additional renewable material.

In a seventh embodiment, the present invention provides a process for producing a renewable material comprising:

(a) charging a reactor with a first amount of hemicellulose-derived material;

(b) converting biologically at least a portion of the first amount of hemicellulose-derived material from step (a) in the reactor for an initial time period of about 1 hour to about 48 hours to form a mixture comprising a renewable material and residual hemicellulose-derived material;

(c) charging a second reactor with the mixture of step (b) after the initial time period, wherein the second reactor contains a second amount of hemicellulose-derived material and lignin; and

(d) converting biologically at least a portion of the second amount of hemicellulose-derived material from step (c) and residual hemicellulose-derived material in the mixture of step (e) to form additional renewable material.

In an eighth embodiment, the present invention provides a process for producing a renewable material comprising:

(a) pretreating a feedstock to provide liquid fraction comprising a hemicellulose-derived material and a solid fraction comprising cellulose and lignin;

(b) separating the liquid fraction from the solid fraction;

(c) converting biologically at least a portion of a hemicellulose-derived material to form a first mixture comprising a first renewable material; and

(d) converting at least a portion of a material comprising cellulose and lignin in the presence of at least a portion of the first mixture to form a second mixture comprising a second renewable material.

In a ninth embodiment, the present invention provides a method of reducing viscosity of a material comprising cellulose and lignin, the method comprising:

(a) converting biologically hemicellulose-derived material to form a mixture comprising a first renewable material; and

(b) combining the material comprising cellulose and lignin with the mixture of step (a).

In a tenth embodiment; the present invention provides a process for producing ethanol from lignocellulosic feedstock comprising:

(a) hydrolyzing in the presence of water the feedstock comprising lignin, cellulose and hemicellulose to form a liquid portion comprising xylose and a solids portion comprising cellulose and lignin;

(b) separating at least a substantial amount of the liquid portion from the solids portion to form a liquid fraction and a solids fraction;

(c) contacting the liquid fraction with a fermentation organism to form a first mixture comprising ethanol;

(d) mixing the solid fraction of step (b) with at least a portion of the first mixture generated in step (c) to form a slurry; and

(e) contacting the slurry of step (d) with an enzyme and the fermentation organism to form a second mixture comprising ethanol.

In an eleventh embodiment, the present invention provides a method of producing renewable material comprising:

(a) detoxifying a hemicellulose-derived material;

(b) converting biologically the detoxified hemicellulose-derived material to form a first mixture comprising a first renewable material; and

(c) converting a material comprising cellulose in the presence of at least a portion of the first mixture to form a second mixture comprising a second renewable material.

In one embodiment, the step of converting in step (c) comprises saccharification of the material comprising cellulose.

In one embodiment, the method further comprises fermentation of the second renewable material formed in step (c).

In one embodiment, fermentation and saccharification occur simultaneously.

In one embodiment, saccharification comprises enzymatic hydrolysis.

In one embodiment, fermentation is performed by a microorganism.

In one embodiment, the microorganism used following step (c) is selected from one or more of Escherichia, Zymomonas, Saccharomyces, Candida, Pichia, Streptomyces, Bacillus Schizosaccharomyces, Dekkera, Bretanomyces, Kluyveromyces, Issatchenkia, Hansenula, Pachysolen, Torulaspora, Zygosaccharomyces, Yarrowia, Lactobacillus, and Clostridium.

In one embodiment, the step of converting in step (b) comprises fermentation.

In one embodiment, the hemicellulose-derived material comprises xylose, xylooligomers and combinations thereof.

In one embodiment, the fermentation of step (b) is performed by a microorganism.

In one embodiment, the microorganism used in step (b) is selected from one or more of Escherichia, Zymomonas, Saccharomyces, Candida, Pichia, Streptomyces, Bacillus, Schizosaccharomyces, Dekkera, Bretanomyces, Kluyveromyces, Issatchenkia, Hansenula, Pachysolen, Torulaspora, Zygosaccharomyces, Yarrowia, Lactobacillus, and Clostridium.

In one embodiment, the first and second renewable materials are the same.

In one embodiment, the first and second renewable materials are selected from the group consisting of alcohols and organic acids.

In one embodiment, the alcohol is selected from the group consisting of ethanol and butanol.

In one embodiment, the microorganism used in step (b) and following step (c) is the same.

In one embodiment, the hemicellulose-derived material and the material comprising cellulose are obtained from hydrolysis of a lignocellulosic feedstock.

In one embodiment, hydrolysis comprises the step of contacting the feedstock with an acid.

In one embodiment, the step of detoxifying in step (a) occurs by adjusting the pH of the hemicellulose-derived material.

In one embodiment, the pH of the hemicellulose-derived material in the range of about 1.5 to about 2.5 is increased to a range of about 5.0 to about 9.0.

In one embodiment, the pH of the hemicellulose-derived material in the range of about 3.0 to about 4.0 is increased to a range of about 5.0 to about 6.0.

In one embodiment, the feedstock is selected from the group consisting of Miscanthus, Erianthus, energy cane, sugar cane, sorghum, Napier grass, and switch grass.

In one embodiment, the first mixture reduces the viscosity of the second mixture.

In one embodiment, step (b) and step (c) occur in the same vessel.

In one embodiment, step (b) and step (c) occur in a different vessel.

In one embodiment, the enzyme for the enzymatic hydrolysis is selected from the group comprising cellobiohydrolase I, cellobiohydrolase II, beta-glucosidase, endoglucanase or any combination thereof.

In one embodiment, the second renewable material is a sugar.

In one embodiment, the sugar comprises glucose.

In a twelfth embodiment, the present invention provides a method for producing an alcohol comprising:

(a) detoxifying a liquid fraction comprising xylose;

(h) fermenting a portion of the xylose in the liquid fraction during a first time to form a first mixture comprising an alcohol and unfermented xylose;

(c) saccharifying a material comprising cellulose to form glucose in a second mixture comprising at least a portion of the first mixture; and

(d) fermenting in the second mixture a portion of the glucose in the second mixture and at least a portion of the unfermented xylose from the first mixture, to form an alcohol.

In one embodiment, the fermenting in step (d) and the saccharifying in step (c) occur simultaneously.

In one embodiment, the fermenting in step (b) and the fermenting in step (d) are in separate vessels.

In one embodiment, the fermenting in step (b) and the fermenting in step (d) are in the same vessel.

In one embodiment, the alcohol is selected from the group consisting of ethanol and a butanol.

In one embodiment, the alcohol is ethanol.

In one embodiment, the butanol is isobutanol.

In a thirteenth embodiment, the present invention provides a method of producing renewable material comprising:

(a) hydrolyzing in the presence of water a feedstock comprising cellulose and hemicellulose to form a liquid portion comprising hemicellulose-derived material and a solids portion comprising cellulose;

(b) separating at least a substantial amount of the liquid portion from the solids portion to forming a liquid fraction and a solids fraction;

(c) detoxifying the liquid portion;

(d) converting biologically in the presence of water at least a portion of the hemicellulose-derived material in the detoxified liquid fraction to form a first mixture comprising water and a first renewable material;

(e) combining at least a portion of the first mixture with at least a portion of the solids fraction to form a second mixture having reduced viscosity relative to the solids fraction; and

(f) converting at least a portion of cellulose in the second mixture to form a second renewable material.

In one embodiment, the solids fraction has a solids content of about 30% to about

In one embodiment, the solids content of the second mixture is about 10% w/v to about 15% w/v, and more specifically 12% w/v to about 14% w/v, and even more specifically about 13.5% w/v.

In a fourteenth embodiment, the present invention provides a method of producing renewable material comprising:

(a) detoxifying a liquid fraction comprising a hemicellulose-derived material;

(b) converting biologically at least a portion of the hemicellulose-derived material to form a first mixture comprising a first renewable material; and

(c) converting biologically at least a portion of a cellulose-derived material in the presence of at least a portion of the first mixture to form a second mixture comprising a second renewable material.

In a fifteenth embodiment, the present invention provides a process for producing a renewable material comprising:

(a) pretreating a feedstock to provide liquid fraction comprising a hemicellulose-derived material and a solid fraction comprising cellulose;

(b) separating the liquid fraction from the solid fraction;

(c) detoxifying the liquid fraction;

(d) converting biologically at least a portion of the hemicellulose-derived material to form a first mixture comprising a first renewable material; and

(e) converting at least a portion of a material comprising cellulose in the presence of at least a portion of the first mixture to form a second mixture comprising a second renewable material.

In a sixteenth embodiment, the present invention provides a process for producing a renewable material comprising:

(a) detoxifying a liquid fraction comprising a first bolus of hemicellulose-derived material;

(b) converting biologically at least a portion of the first bolus of the hemicellulose-derived material for an initial time period of about 1 hour to about 48 hours to form a mixture comprising a renewable material and residual hemicellulose-derived material;

(c) adding a second bolus of a hemicellulose-derived material after the initial time period; and

(d) converting biologically the at least a portion of the second bolus of hemicellulose-derived material from step (c) and residual hemicellulose derived material from step (b) to form additional renewable material.

In a seventeenth embodiment, the present invention provides a method of reducing viscosity of a material comprising cellulose, the method comprising:

(a) detoxifying a liquid fraction comprising a hemicellulose-derived material;

(b) converting biologically the hemicellulose-derived material to form a mixture comprising a first renewable material; and

(c) combining the material comprising cellulose with the mixture of step (b).

In an eighteenth embodiment, the present invention provides a process for producing ethanol from lignocellulosic feedstock comprising:

(a) hydrolyzing in the presence of water the feedstock comprising cellulose and hemicellulose to form a liquid portion comprising xylose and a solids portion comprising cellulose;

(b) separating at least a substantial amount of the liquid portion from the solids portion to form a liquid fraction and a solids fraction;

(c) detoxifying the liquid fraction;

(d) contacting the liquid fraction of step (c) with a fermentation organism to form a first mixture comprising ethanol;

(e) mixing the solid fraction of step (b) with at least a portion of the first mixture generated in step (d) to form a slurry; and

(f) contacting the slurry of step (e) with an enzyme and the fermentation organism to form a second mixture comprising ethanol.

In a nineteenth embodiment, the present invention provides a process for producing ethanol from lignocellulosic feedstock comprising:

(a) hydrolyzing in the presence of water the feedstock comprising lignin, cellulose and hemicellulose to form a liquid portion comprising xylose and a solids portion comprising cellulose and lignin;

(b) separating at least a substantial amount of the liquid portion from the solids portion to form a liquid fraction and a solids fraction;

(c) detoxifying the liquid fraction;

(d) contacting the liquid fraction of step (c) with a fermentation organism to form a first mixture comprising ethanol;

(e) mixing the solid fraction of step (b) with at least a portion of the first mixture generated in step (d) to form a slurry; and

(f) contacting the shiny of step (e) with an enzyme and the fermentation organism to form a second mixture comprising ethanol.

In a twentieth embodiment, the present invention provides a process for producing ethanol from lignocellulosic feedstock comprising:

(a) hydrolyzing in the presence of water the feedstock comprising lignin, cellulose and hemicellulose to form a liquid portion comprising xylose and a solids portion coinprising cellulose and lignin;

(b) separating at least a substantial amount of the liquid portion from the solids portion to form a liquid fraction and a solids fraction;

(c) detoxifying, the liquid fraction;

(d) contacting the liquid fraction of step (c) with a fermentation organism to form a first mixture comprising ethanol;

(e) heating the first mixture to a temperature to inactivate the fermentation organism;

(f) mixing the solid fraction of step (b) with at least a portion of the heat treated first mixture generated in step (e) to form a slurry; and

(g) contacting the slurry of step (f) with an enzyme and the fermentation organism to form a second mixture comprising ethanol.

### BRIEF DESCRIPTION OF THE DRAWINGS

The particular features and advantages of the invention as well as other aspects will become apparent from the following description taken in connection with the accompanying drawings, in which:

FIG. 1A is a schematic showing a process flow diagram of one embodiment of the present invention;

FIG. 1B is a schematic showing a process flow diagram of an alternative embodiment of the present invention;

FIG. 2 is a schematic showing steps of the continuous detoxification process including flowing a first continuous stream of a hydrolysate into a continuous reactor, flowing a second continuous stream of a solution of a magnesium base into the continuous reactor, mixing the hydrolysate with the magnesium base in the continuous reactor for a period of time sufficient to reduce the quantity of toxins in the hydrolysate, and flowing the hydrolysate out of the continuous reactor;

FIGS. 3A and 3B show ethanol production and xylose consumption using two different S. cerevisiae yeast strains in the primary fermentation; and

FIGS. 4A and 4B show ethanol production and xylose consumption using two different S. cerevisiae yeast strains in the secondary fermentation.

### DETAILED DESCRIPTION OF THE INVENTION

The invention is described with reference to the drawings in which like elements are referred to by like numerals. The relationship and functioning of the various elements of this invention are better understood by the following detailed description. However, the embodiments of this invention as described below are by way of example only, and the invention is not limited to the embodiments illustrated in the drawings. It should also be understood that the drawings are not to scale and in certain instances details have been omitted, which are not necessary for an understanding of the present invention, such as conventional details of fabrication and assembly.

### DEFINITIONS

As used herein, the term “renewable material” preferably refers to a substance and/or an item that has been at least partially derived from a source and/or a process capable of being replaced at least in part by natural ecological cycles and/or resources. Renewable materials may broadly include, for example, chemicals, chemical intermediates, solvents, adhesives, lubricants, monomers, oligomers, polymers, biofuels, biofuel intermediates, biogasoline, biogasoline blendstocks, biodiesel, green diesel, renewable diesel, biodiesel blend stocks, biodistillates, biochar, biocoke, renewable building materials, and/or the like. In certain embodiments, the renewable material may include one or more biofuel components. For example, the renewable material may include an alcohol, such as ethanol, butanol, or isobutanol, or lipids.

Lignocellulosic preferably broadly refers to materials containing cellulose, hemicellulose, lignin, and/or the like, such as may be derived from plant material and/or the like. Lignocellulosic material may include any suitable material, such as sugar cane, sugar cane bagasse, energy cane bagasse, rice, rice straw, corn, Arundo donax, corn stover, wheat, wheat straw, maize, maize stover, sorghum, sorghum stover, sweet sorghum, sweet sorghum stover, cotton remnant, sugar beet, sugar beet pulp, soybean, rapeseed, jatropha, switchgrass, miscanthus, other grasses, cacti, timber, softwood, hardwood wood waste, sawdust, paper, paper waste, agricultural waste, municipal waste, any other suitable biomass material, and/or the like.

Lignin preferably broadly refers to a biopolymer that may be part of secondary cell walls in plants, such as a complex highly cross-linked aromatic polymer that may covalently link to hemicellulose.

Hemicellulose preferably broadly refers to a branched sugar polymer composed mostly of pentoses, such as with a generally random amorphous structure and typically may include up to hundreds of thousands of pentose units.

Cellulose preferably broadly refers to an organic compound with the formula (C₆H₁₀O₅)_{z} where z includes any suitable integer. Cellulose may include a polysaccharide with a linear chain of several hundred to over ten thousand hexose units and a high degree of crystalline structure, for example.

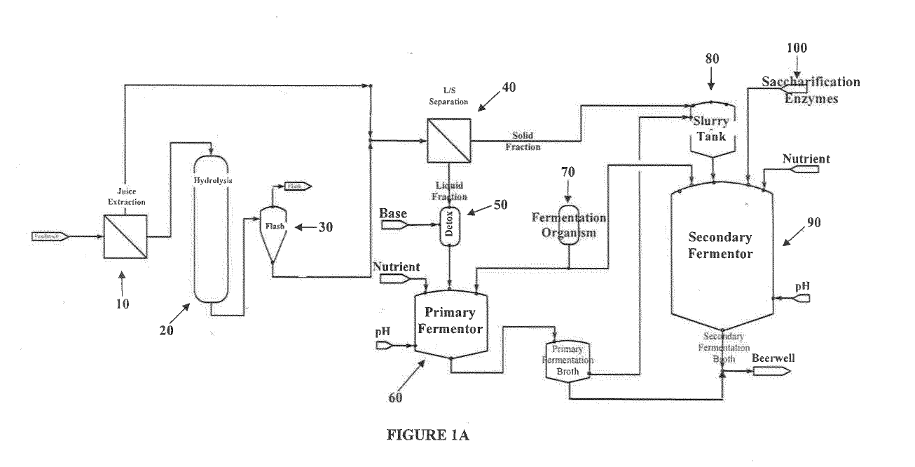

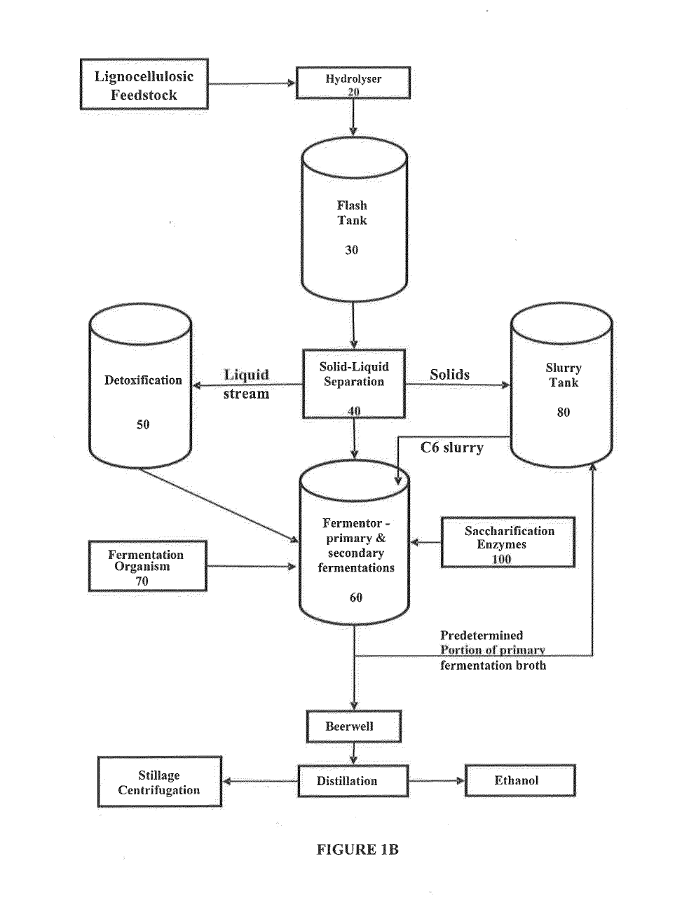

Turning now to the drawings, and more particularly to FIG. 1A, what is shown is lignocellulosic feedstock mechanically prepared using a dewatering unit 10 such as, for example, a belt press or roller mill to control moisture content of the biomass prior to pretreatment.

The pressed feedstock is fed to a pretreatment reactor 20 and contacted with steam and dilute acid under positive pressure to solubilize and hydrolyze a portion of the hemicellulose to form, among other things, soluble sugars such as xylose, xylobiose, and other xylooligomers (“hemicellulose-derived material”). Dilute acids can be selected from the group comprising sulfuric acid, nitric acid, phosphoric acid, acetic acid, citric acid, perchloric acid, hydroiodic acid, hydrobromic acid, hydrofluoric acid, formic acid, hydrocyanic acid and any mixture or combination thereof.

Alternatively, the pressed feedstock can undergo auto-hydrolysis by being fed to a pretreatment reactor at atmosphere or under positive pressure conditions and contacted with water that has been heated to solubilize, and in some instances hydrolyze, a portion of the cellulose and hemicellulose.

The pressurized pretreated biomass is then rapidly released from the pretreatment reactor (“flashed”) into a blow cyclone 30 which is at a pressure in the range of about atmospheric pressure to about 20 psi. The sudden change in pressure causes the cellulose fibers to mechanically rupture making them more accessible for enzymatic saccharification. Upon discharge front the pretreatment reactor the insoluble cellulosic fiber solids fraction is separated front the liquid fraction using one or more separation devices 40 for separating solids form liquids, such as filters, presses, such as screw presses, centrifuges and the like.

In the case of dilute acid pretreatment, the pH of the liquid fraction is adjusted using a caustic agent in vessel or reactor 50. This step of pH adjustment also serves to substantially, detoxify the liquid fraction by removing the acidic and phenolic compounds are potentially toxic to a fermentation organism or have an inhibitory effect during the fermentation process. In one embodiment, the pH adjusted and substantially detoxified hydrolysate is fermented in a primary fermentor 60 using a yeast based fermentation organism which is propagated in a separate propagation in vessel or reactor 70 to provide a primary fermentation broth (“a first mixture”). In one embodiment, the caustic agent is selected from the group consisting essentially of sodium hydroxide, ammonia, ammonium hydroxide, magnesium hydroxide, calcium hydroxide, ammonia, potassium hydroxide, barium hydroxide, cesium hydroxide, strontium hydroxide, lithium hydroxide, rubidium hydroxide, and any mixture or combination thereof.

Following primary fermentation of the soluble sugars, a portion of the primary fermentation broth containing ethanol (“a first renewable material”) is added to the solids fraction (“material comprising cellulose and lignin”) which contains lignin and cellulose fiber to form slurry in a slurry tank 80. The slurry stream is subsequently fed to a secondary fermentor 90. Saccharification enzymes which are propagated in vessel or reactor 100 are added to the slurry in secondary fermentor 90 and the cellulose fiber undergoes saccharification. If fermentation organism is added to the secondary fermentor concurrently with the cellulase enzymes than fermentation can occur simultaneously converting the liberated sugars to ethanol (“a second renewable material”).

In another embodiment, shown in FIG. 1B the slurry from slurry tank 80 is transferred to fermenter 60 in which the primary fermentation occurred. That is, the primary and secondary fermentations utilize the same tank 60 in sequential order.

After the fermentation is complete the ethanol containing mixture is sent to product recovery. Recovery consists of distilling ethanol from the mixture in a distillation column followed by a rectification column. The ethanol-water azeotropic mixture from the rectification column is passed through molecular sieves to provide substantially anhydrous ethanol. The stillage from the bottom of the distillation column is processed to separate the lignin containing solids.

It has now been discovered that the lignin is extremely beneficial to an ethanol producer. Lignin can be fired in a boiler to produce electricity. Lignin possesses high BTU content and is clean burning resulting in lower carbon emissions than those produced by homing coal.

It has also been discovered theta delignification step following liquid-solid separation in our process would in fact be detrimental because it would result in a high loss of residual hemicellulose sugars and residual sucrose entrained in the solids.

In one embodiment, the lignin solids are fed to a boiler which generates steam from a portion of liquid sent to waste water treatment. The remaining waste water is treated anaerobically to generate biogas. The biogas from the waste water treatment process is fed to the boiler where it is burned along with lignin containing solids to generate steam and electricity.

It has also been discovered that detoxification of the liquid stream reduces the amount of toxic compounds that can inhibit a fermentation organism. Therefore, less of the fermentation organism needs to be utilized throughout the process.

It has also been discovered that the overall water balance of the process can be reduced by utilizing the primary fermentation broth to slurry the solids instead of water,

### Feedstock

The term “feedstock,” as used herein, is lignocellulosic, referred to herein as a lignocellulosic feedstock, comprises cellulose, which is a polymer of glucose linked by β-1,4-glucosidic bonds, hemicellulose, which is a polysaccharide composed of different five-carbon pentose sugars and six-carbon hexose sugars linked by variety of different β and α linkages, and lignin, which is a complex polymer consisting of phenyl propane units linked by ether or carbon-carbon bonds.

Feedstock which can be hydrolyzed according to the methods of this disclosure can include agricultural crops, plant waste, or byproducts of food processing or industrial processing such as, for example, grasses, wood, seeds, grains, corn stalks, corn byproducts, Arundo donax, corn stover, corn fiber, corn cobs, corn husks, grass, bagasse, such as sugar cane bagasse and energy cane bagasse, straw, for example, straw from rice, wheat, buckwheat, amaranth, rye, millet, oat, barley, rape, sorghum, spelt straw, wood, including wood chips, wood bark, wood saw dust, and other wood byproducts, wood waste and wood processing waste, where the wood, chips, bark, sawdust and other wood byproducts, wood waste and wood processing waste can be deciduous or coniferous wood, hardwood or softwood, paper and paper byproducts, paper pulp, paper waste, paper mill waste, and recycled paper such as recycled newspaper, recycled printer paper, and the like. Other feedstock materials include, without limitation, soybean, rapeseed, barley, rye, oats, wheat, sorghum, sudan, milo, bulgur, rice, forest residue, and agricultural residue. Feedstock which can be hydrolyzed according to the methods of this disclosure can include tubers, for example, beets, such as sugar beets, and potatoes.

The lignocellulosic feedstock is suitably grass and plants from the grass family. The proper name is the family known as Poaceae or Gramineae in the class Liliopsida (the monocots) of the flowering plants. Plants of this family are usually called grasses, and include bamboo. There are about 600 genera and some 9,000 to 10,000 or more species of grasses (Kew Index of World Grass Species).

Poaceae includes the staple food grains and cereal crops grown around the world, lawn and forage grasses, and bamboo.

The success of the grasses is believed to lie in part in their morphology and growth processes, and in part in their physiological diversity. Most of the grasses divide into two physiological groups, using the three carbon (C3) and four carbon (C4) photosynthetic pathways for carbon fixation. The C4 grasses have a photosynthetic pathway linked to specialized leaf anatomy that particularly adapts them to hot climates and an atmosphere low in carbon dioxide. C3 grasses are referred to as “cool season grasses” while C4 plants are considered “warm season grasses.”

Grasses may be annual or perennial. Examples of annual cool season grasses are wheat, rye, annual bluegrass such as annual meadowgrass, Poa annua and oat. Examples of perennial cool season are orchardgrass, such as cock's foot (Dactylis glomerata), fescue (Festuca spp.), Kentucky bluegrass and perennial ryegrass (Lolium perenne). Examples of annual warm season grasses are corn, sudangrass and pearl millet. Examples of perennial warm season grasses are big bluestem, indiangrass, bermudagrass and switchgrass.

One classification of the grass family recognizes twelve subfamilies, all of which can be feedstock in embodiments of this invention: These are 1) anomochlooideae, a small lineage of broad-leaved grasses that includes two genera (Anomochloa, Streptochaeta); 2) Pharoideae, also known as Poaceae, a small lineage of grasses that includes three genera, including Pharus and Leptaspis; 3) Puelioideae, a small lineage that includes the African genus Puelia; 4) Pooideae, which includes wheat, barley, oats, brume-grass (Bromus) and reed-grasses (Calamagrostis); 5) Bambusoideae, which includes bamboo; 6) Ehrhartoideae, which includes rice, and wild rice; 7) Arundinoideae, which includes the giant reed and common reed 8) Centothecoideae, a small subfamily of 11 genera that is sometimes included in Panicoideae; 9) Chloridoideae, including the lovegrasses (Eragrostis, ca. 350 species, including teff), dropseed grasses (Sporobolus, some 160 species), finger millet (Eleusine coracana (L.) Gaertn.), and the rashly grasses (Muhlenbergia, ca. 175 species); 10) Panicoideae including panic grass, maize, sorghum, sugar cane, most millets, fonio and bluestem grasses; 11) Micrairoideae; 12) Danthoniodieae, including pampas grass; with Poa which is a genus of about 500 species of grasses, native to the temperate regions of both hemisphere.

Agricultural grasses grown for their edible seeds are called cereals. Three common cereals are rice, wheat and maize (corn). Of all crops, 70% are grasses.

A suitable feedstock is selected from the group consisting of the energy crops. In a further embodiment, the energy crops are grasses. Suitable grasses include Napier Grass or Uganda Grass, such as Pennisetum purpureum; or, Miscanthus; such as Miscanthus giganteus and other varieties of the genus miscanthus, or Indian grass, such as Sorghastrum nutans; or, switchgrass, for example, as Panicum virgatum or other varieties of the genus Panicum, giant reed (arundo donax.), energy cane (saccharum spp.). In some embodiments the feedstock is sugarcane, which refers to any species of tall perennial grasses of the genus Saccharum.

Other suitable types of feedstock include quinoa, anile stubble, citrus waste, urban green waste or residue, food manufacturing industry waste or residue, cereal manufacturing waste or residue, hay, grain cleanings, spent brewer's grain, rice hulls, calix, spruce, poplar, eucalyptus, Brassica carinata residue, Antigonum leptopus, sweetgum, Sericea lespedeza, Chinese tallow, hemp, Sorghum bicolor, soybeans and soybean products such as, for example, soybean leaves, soybeans stems, soybean pods, and soybean residue, sunflowers and sunflower products, such as, for example, leaves, sunflower stems, seedless sunflower heads, sunflower hulls, and sunflower residue, Arundo, nut shells, deciduous leaves, cotton fiber, manure, coastal Bermuda grass, clover, Johnsongrass, flax, amaranth and amaranth products such as, for example, amaranth stems, amaranth leaves, and amaranth residue and alfalfa.

For wood as a feedstock, the feedstock includes hardwood and softwood. Examples of suitable softwood and hardwood trees include, but are not limited to, the following: pine trees, such as loblolly pine, jack pine, Caribbean pine, lodgepole pine, shortleaf pine, slash pine, Honduran pine, Masson's pine, Sumatran pine, western, white pine, egg-cone pine, longleaf pine, patula pine, maritime pine, ponderosa pine, Monterey pine, red pine, eastern white pine, Scots pine, araucaria tress; fir trees, such as Douglas fir; and hemlock trees, plus hybrids of any of the foregoing. Additional examples include, but are not limited to, the following: eucalyptus trees, such as Dunn's white gum, Tasmanian, blue gum, rose guru, Sydney blue gum, Timor white gum, and the E. urograndis hybrid; populus trees, such as eastern cottonwood, bigtooth aspen, quaking aspen, and black cottonwood; and other hardwood trees, such as red alder, Sweetgum, tulip tree, Oregon ash, green ash, and willow, plus hybrids of any of the foregoing.

The feedstock can be one or more of, for example, Miscanthus floridulus, Miscanthus giganteus, Miscanthus sacchariflorus, Miscanthus sinensis, Miscanthus tinctorius, Miscanthus transmorrisonensis, Erianthus, such as, E. acutecarinatus, E. acutipennis -E. adpressus, E. alopecuroides, E. angulatus, E. angustifolius, E. armatus, E. articulatus, E. arundinaceus, E. asper, E. aureus, E. bakeri, E. balansae, E. beccarii, E. bengalensis, E. biaristatus, E. bifidus, E. birmanicus, E. bolivari, E. brasilianus, E. brevibarbis, E. capensis, E. chrysothrix, E. ciliaris, E. clandestinus, E. coarctatus, E. compactus, E. contortus, E. cumingii, E. cuspidatus, E. deccus-sylvae, E. deflorata, E. divaricatus, E. dohrni, E. ecklonii, E. elegans, E. elephantinus, E. erectus, E. fallax, E. fastigiatus, E. filifolius, E. fischerianus, E. flavescens, E. flavipes, E. flavoinflatus, E. floridulus, E. formmosanus, E. formosus, E. fruhstorferi, E. fulvus, E. giganteus, E. glabrinodis, E. glaucus, E. griffithii, E. guttatus, E. hexastachyus, E. hookeri, E. hostii, E. humbertianus, E. inhamatus, E. irritans, E. jacquemontii, E. jamaicensis, E. japonicus, E. junceus, E. kajkaiensis, E. kanashiroi, E. lancangensis, E. laxus, E. longesetosus, E. longifolius, E. longisetosus, E. longisetus, E. lugubris, E. luzonicus, E. mackinlayi, E. macratherus, E. malcolmi, E. manueli, E. maximus, E. mishmeensis, E. mollis, E. monstierii, E. munga, E. munja, E. nepalensis, E. nipponensis, E. nudipes, E. obtusus, E. orientalis, E. pallens, E. parviflorus, E. pedicellaris, E. perrieri, E. pictus, E. pollinioides, E. procerus, E. pungens, E. purpurascens, E. purpureus, E. pyramidalis, E. ravennae, E. rehni, E. repens, E. rockii, E. roxburghii, E. ruflpilus, E. rufus, E. saccharoides, E. sara, E. scriptorius, E. sesquimetralis, E. sikkimensis, E. smallii, E. sorghum, E. speciosus, E. strictus, E. sukhothaiensis, E. sumatranus, E. teretifolius, E. tinctorius, E. tonkinensis, E. tracyi, E. trichophyllus, E. trinii, E. tristachyus, E. velutinus, E. versicolor, E. viguieri, E. villosus, E. violaceus, E. vitalisi, E. vulpinus, E. wardii, E. williamsii; energy cane, such as sugar cane, for example, S. acinaciforme, S. aegyptiacum, S. alopecuroides, S. alopecuroideum, S. alopecuroidum, S. alopecurus, S. angustifoliumn, S. antillarum, S. appressum, S. arenicola, S. argenteum, S. arundinaceum, S. asperum, S. atrorubens, S. aureum, S. balansae, S. baldwini, S. baldwinii, S. barberi, S. barbicostatum, S. beccarii, S. bengalense, S. benghalense, S. bicome, S. biflorum, S. boga, S. brachypogon, S. bracteatum, S. brasilianum, S. brevibarbe, S. brevifolium, S. brunneum, S. caducum, S. caffrosum, S. canaliculatum, S. capense, S. casi, S. caudatum, S. cayennense, S. chinense, S. ciliare, S. coarctatum, S. confertum, S. conjugatum, S. contortum, S. contractum, S. cotuliferum, S. cylindricum, S. deciduum, S. densum, S. diandrum. S. dissitiflorum, S. distichophyllum, S. dubium, S. ecklonii, S. edule, S. elegans, S. elephantinum, S. erianthoides, S. europaeum, S. exaltatum, S. fallax, S. fasciculatum, S. fastigiatum, S. fatuum, S. filifolium, S. filiforme, S. floridulum. S. formosanum, S. fragile, S. fulvum, S. fuscun, S. giganteum, S. glabrum, S. glaga, S. glaucum, S. glaza, S. grandiflorum, S. griffithii, S. hildebrandtii, S. hirsutum, S. holcoides, S. hookeri, S. hybrid, S. hybridum, S. indum, S. in[beta]mum, S. insulare, S. irritans, S. jaculatorium, S. jamaicense, S. japonicum, S. juncifolium, S. kajkaiense, S. kanashiroi, S. klagha, S. koenigii, S. laguroides, S. longifolium, S. longisetosum, S. longisetum, S. lota, S. luzonicum, S. macilentum, S. macrantherum, S. maximum, S. mexicanum, S. modhara, S. modhua, S. monandrum, S. moonja, S. munja, S. munroanum, S. muticum, S. narenga, S. nareya, S. negrosense, S. obscurum, S. occidentale, S. officinale, S. officinalis, S. officinarum, S. palisoti, S. pallidum, S. paniceum, S. panicosum, S. pappiferum, S. parviflorum, S. pedicellare, S. perrieri, S. polydactylum, S. polystachyon, S. polystachyum, S. porphyrocomum, S. praegrande, S. procerum, S. propinquum, S. punctatum, S. purpuratum, S. rara, S. rarum, S. ravennae, S. repens, S. reptans, S. revennac, S. ridleyi, S. robustum, S. roseum, S. rubicundum, S. ru[beta]pilum, S. rufum, S. sagittatum, S. sanguineum, S. sape, S. sara, S. sarpata, S. scindicus, S. semidecumbens, S. seriferum, S. sibiricum, S. sikkimense, S. sinense, S. sisca, S. soltwedeli, S. sorghum, S. speciosissimum, S. sphacelaturn, S. spicatum, S. spontaneum, S. spontaneum, S. stenophyllum, S. stewartii, S. strictum, S. teneriffae, S. tenuius, S. ternatum, S. thunbergii, S. tinctorium, S. tridentatum, S. trinii, S. tripsacoides, S. tristachyum, S. velutinum, S. versicolor, S. viguieri, S. villosum, S. violaceum, S. wardii, S. warmingianum, S. williamsii; hybrids, for example, L 99-233, L 99-226, L79-1001, L 79-1002, L 99-233, L 99-226, HoCP 91-552, HoCP 91-555, Ho 00-961, Ho 02-113, Ho 03-19, Ho 03-48, Ho 99-51, Ho 99-58, US 72-114, Ho 02-144, Ho 06-9002; a sorghum, such as, Sorghum almum, Sorghum amplum, Sorghum angustum, Sorghum arundinaceum, Sorghum bicolor, Sorghum bicolor subsp, drummondii—Sudan grass, Sorghum brachypodum, Sorghum bulbosum, Sorghum burmahicum, Sorghum controversum, Sorghum drummondii, Sorghum ecarinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum propinquum, Sorghum purpureosericeum, Sorghun stipoideum, Sorghum timorense, Sorghum trichocladum, Sorghum versicolor, Sorghum virgatum, Sorghum vulgare, hybrids, such as sugar cane×Miscanthus or sugar cane×Erianthus; Napier grass (elephant grass), for example, Pennisetum purpureum; or switch grass, for example, Panicum virgatum.

The lignocellulosic feedstock can be on or more of rice, stover, wheat, maize, maize stover, sorghum, sorghum stover, sweet sorghum, sweet sorghum stover, cotton, cotton remnant, cassaya, sugar beet pulp, soybean, rapeseed, jatropha, switchgrass, miscanthus, other grasses, timber, agricultural waste, manure, dung, sewage, municipal solid waste, any other suitable feedstock material, and/or the like.

The feedstock can be used either in a green state, that is feedstock that is freshly harvested from the farm or plantation where it is grown, or it can be aged and at least partially dried or completely dried.

### Feedstock Pretreatment

Washed, milled fiber feedstock is hydrolyzed in an acid hydrolysis reactor 20 shown in FIGS. 1A and 1B. A majority of the hemicellulose polymer is converted into a mixture of pentose monomers (xylose) and oligomers, with some further degradation of pentose sugars to undesired toxins such as furfural. In addition, a much smaller portion of the cellulose polymer is also converted into a mixture of hexose nmonomers (glucose) and oligomers.

In some cases, feedstocks, such as lignocellulosic feedstocks, are subject to dilute acid hydrolysis during which hemicellulose is hydrolyzed to monomeric sugars producing a liquid stream containing the sugars and the crystalline structure of cellulose is damaged, facilitating future enzymatic digestion (solid fiber). The liquid containing pentose and hexose sugars, so called hydrolysate, is separated from cellulose and lignin solids can be fermented to various products such as ethanol. In addition to sugars however, hydrolysate can also contain aliphatic acids, esters (acetate), phenolics that are different compounds obtained from lignin hydrolysis, and products of sugar dehydration, including the furan aldehydes, furfural and 5-hydroxymethyl furfural (5-HMF) and other compounds. Most of these compounds have a negative impact on microorganisms and can inhibit fermentation of sugars to an alcohol such as ethanol. Detoxification of the hydrolysate prior to fermentation is one measure that can be taken in order to avoid inhibition caused by toxic compounds present in the hydrolysate.

Any suitable hydrolysis process can be used to prepare hydrolysates, including acid hydrolysis and base hydrolysis. Acid hydrolysis is a relatively inexpensive and can be a fast method and can suitably be used. A concentrated acid hydrolysis is suitably operated at temperatures of about 20° C. to about 100° C., and an acid strength in the range of about 10% to about 45% (e.g., about 10%, 10.5%, 11%, 11.5%, 12%, 12.5%, 13%, 13.5%, 14%, 14.5%, 15%, 15.5%, 16%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 37%, 37.5%, 38%, 38.5%, 39%, 39.5%, 40%, 41%, 41.5%, 42%, 42.5%, 43%, 43.5%, 44%, 44.5%, 45% or any range bounded by any two of the foregoing values). Dilute acid hydrolysis is a simpler process, but is optimal at higher temperatures, for example at about 80° C. to about 230° C., and generally higher pressure.

Different kinds of acids, with concentrations in the range of about 0.001% to about 10%, for example, about 0.001%, 0.01%, 0.05%, 0.1%, 0.15%, 0.2%, 0.25%, 0.3%, 0.35%, 0.4%, 0.5%, 0.6%, 0.7%, 0.8%, 0.9%, 1%, 2%, 3%, 4%, 5%, 5.5%, 6%, 6.5%, 7%, 7.5%, 8%, 8.5%, 9%, 9.5% or 10%, or any range bounded by any two of the foregoing values, can be used. Suitable acids, for either the concentrated or dilute hydrolysis includes, for example, nitric acid, sulfurous acid, nitrous acid, phosphoric acid, acetic acid, hydrochloric acid and sulfuric acid. Sulfuric acid is a particularly useful acid to use for the hydrolysis step.

Depending on the acid concentration, and the temperature and pressure under which the acid hydrolysis step is carried out, corrosion resistant equipment and/or pressure tolerant equipment may be needed.

The hydrolysis can be carried out for a time period ranging from about 2 minutes to about 10 hours, for example, about 2, 3, 4, 5, 6, 7, 8, 9, 10, 15, 20, 25, 26, 27, 28, 29, or 30 minutes, or about 0.5, 0.75, 1, 1.5, 2, 2.5, 3, 3.5, 4, 4.5, 5, 5.5, 6, 6.5, 7, 7.5, 8, 8.5, 9, 9.5 or 10 hour, or range bounded by any two of the foregoing values. For example, the time period for the hydrolysis can be about 1 minute to about 2 hours, about 2 minutes to about 15 minutes, about 2 minutes to about 2 hours, about 15 minutes to about 2 hours, about 30 minutes to about 2 hours, about 10 minutes to about 1.5 hours, or about 1 hour to about 5 hours.

The hydrolysis can also include, either with or without an acid treatment, and either before or after such acid treatment, a heat or pressure treatment or a combination of heat and pressure, for example, treatment with steam, for about 0.5 hours to about 10 hours, for example, about 0.5, 1, 1.5, 2, 2.5, 3, 3.5, 4, 4.5, 5.5, 6, 6.5, 7, 7.5, 8, 8.5, 9, 9.5 or 10 hours, or any range bounded by any two of the foregoing values.

Variations of acid hydrolysis methods are known in the art. For instance, the hydrolysis can be carried out by subjecting the feedstock to a two step process. The first is a chemical hydrolysis step suitably carried out in an aqueous medium at a temperature and a pressure chosen to effectuate primarily depolymerization of hemicellulose without achieving significant depolymerization of cellulose into glucose. This step yields a slurry in which the resulting liquid aqueous phase contains dissolved monosaccharides and soluble and insoluble oligomers of hemicellulose resulting from depolymerization of hemicellulose, and a solid phase containing cellulose and, if present in the feedstock, lignin. See, for example, U.S. Pat. No. 5,536,325. In one embodiment, sulfuric acid is utilized to affect the first hydrolysis step. After the sugars are separated from the first-stage hydrolysis process, the second hydrolysis step is run under more severe condition to hydrolyze the more resistant cellulose fractions.

In another embodiment, the hydrolysis method entails subjecting feedstock material to a catalyst comprising a dilute solution of a strong acid and a metal salt in a reactor. The feedstock can be green or dried. This type of hydrolysis can lower the activation energy, or the temperature, of cellulose hydrolysis, ultimately allowing higher yields of fermentable sugars. See, e.g., U.S. Pat. Nos. 6,660,506; 6,423,145.

A further exemplary method involves processing a lignocellulosic feedstock by one or more stages of dilute acid hydrolysis using about 0.4% to about 2% of an acid; followed by treating the unreacted solid lignocellulosic component of the acid hydrolyzed material with alkaline delignification. See, for example, U.S. Pat. No. 6,409,841. Another exemplary hydrolysis method comprises prehydrolyzing feedstock such as a lignocellulosic feedstock, in a prehydrolysis reactor, adding an acidic liquid to the solid lignocellulosic feedstock to make a mixture; heating the mixture to reaction temperature; maintaining reaction temperature for a period of time sufficient to fractionate the lignocellulosic feedstock into a solubilized portion containing at least about 20% of the lignin from the lignocellulosic feedstock, and a solid fraction containing cellulose; separating the solubilized portion from the solid fraction, and removing the solubilized portion while at or near reaction temperature; and recovering the solubilized portion.

Hydrolysis can also comprise contacting a feedstock with stoichiometric amounts of sodium hydroxide and ammonium hydroxide at a very low concentration. See Teixeira et al., 1999, Appl. Biochem. and Biotech. 77-79:19-34. Hydrolysis can also comprise contacting a lignocellulosic feedstock with a chemical, for example, a base, such as sodium carbonate or potassium hydroxide, at a pH of about 9 to about 14 at moderate temperature and pressure. See PCT Publication WO 2004/081185.

Ammonia hydrolysis can also be used. Such a hydrolysis method comprises subjecting a feedstock to low ammonia concentration under conditions of high solids. See, for example, U.S. Patent Publication No. 20070031918 and PCT publication WO 2006/110901.

In one embodiment, the milled and washed feedstock is partially hydrolyzed thereby converting most or all of the hemicellulose polymers to, primarily, pentose sugars, such as xylose, and oligomeric materials. Some of the cellulose polymer is also converted to hexose sugars, such as glucose. The ratio of water to solids in the hydrolysis reaction can be about 2 to 1. The temperature of the hydrolysis step can be about 135° C. to about 165° C. The reaction time can be about 20 minutes to about 45 minutes. If acid is used as the promoter or catalyst for the hydrolysis, the amount of acid can be about 0.25% w/w to about 0.50% w/w. The pH can be in the range of about 0.5 to about 2.5. The pressure is in the range of about 10 psi to about 200 psi.

The hydrolysis can be accomplished using a number of different procedures and apparatus, such as in a stirred reaction vessel or in a plug flow reactor having vanes or baffles to promote agitation and establish good contact between an acidic or basic aqueous phase and the polymeric sugars in the feedstock. The hydrolysis reaction can be a batch process or a continuous process. It can be single stage or multiple stages, such as 2 or 3 or 4 stages of hydrolysis.

In one embodiment, the comminuted, e.g., shredded, feedstock is treated with steam to add water and elevate the temperature of the feedstock, for example, to a temperature in the range of about 135° C. to about 165° C. The steam treated feedstock is conveyed to a plug-screw feeder where it forms a cake within the feeder. The plug-screw feeder compresses the cake of shredded feedstock into a plug at the end of the screw where it may be treated with acid for the hydrolysis. For example an aqueous acid mixture can be sprayed onto and injected into the plug of feedstock using one or more devices such as nozzles, jets, spray bars or spray rings, and the like. The feedstock so treated is moved down through, for example, a vertical hydrolyser apparatus at a desired rate where it is heated to undertake the hydrolysis reaction as described above. The desired residence time in the vertical hydrolyser unit can be controlled, for example, using a conveying screw on the bottom of the hydrolyser unit. Water can be added to the hydrolyzer to achieve the desired ratio of water to solids in the hydrolysis reaction. The product from the hydrolysis reaction in the hydrolyser unit can be removed from the hydrolyser unit through a valve, orifice or nozzle, for example, at the bottom or lower portion of the hydrolyzer unit. At this stage, the hydrolysis reaction mixture is at an elevated pressure such as for example, a pressure of about 10 psi to about 225 psi. When released, the mixture can undergo a rapid depressurization resulting in what can be referred to a steam explosion where by the particle size of the solids portion of the feedstock material is reduced further. The rapid depressurization can for example, occur within one or more devices such as a blow cyclone. The depressurization can be to atmospheric pressure.

In another embodiment, single stage hydrolysis begins with washed, milled fiber feedstock conveyed from the storage silo to the pre-steaming vessel, where it is conveyed into the plug-screw feeder. The plug-screw feeder compresses the cake into a plug towards the end of the screw. A blowback damper extends to seal the plug pipe to provide pressure isolation should be plug integrity be compromised. At the plug-screw feeder discharge, an acid spray ring distributes dilute acid (3 to 8 weight % sulfuric acid, depending on liquidlsolid ratio target in the hydrolyzer) onto the fiber. Acid-injected fiber is then conveyed down through the vertical hydrolyzer unit, and at the discharge is conveyed via a valve, orifice or nozzle into the blow-cyclone for rapid depressurization/steam explosion. The target liquid/solid ratio, including all condensed steam, in the vertical hydrolyzer is about 2 to 1. Optimum conditions for the singe stage hydrolysis are about 0.25 to about 1.0 weight % acid, about 135° C. to about 165° C. temperature, and about 20 to about 45 minutes residence time. The residence time in the hydrolyser can be controlled by maintaining the height of the biomass bed in the vessel.

### Liquid/Solid Separation

The mixture of solids and liquids produced by the hydrolysis reaction, either with or without the rapid depressurization step, can be treated to separate the liquid from the solid portion thereof using one or more separation devices for separating solids form liquids, such as filters, presses, such as screw presses, centrifuges and the like shown as 40 in FIGS. 1A and 1B. For example, one or more, such as 2, 3 or 4 such separation devices can be arranged in series where the solids containing portion from the first device is sent to the second device in series, and so on, until the desired separation of the liquid portion from the solid portion is achieved. For example, at least about 50 percent of the water in the mixture of solids and liquids produced by the hydrolysis reaction is removed by the separation process, or at least about 60 percent of the water, or at least about 70 percent, or at least about 80 percent of the water is removed. The water that is removed can contain at least about 50 percent of the soluble sugars, such as one or more of sucrose, glucose, fructose, and xylose, at least about 60 percent, or at least about 70 or 80 percent, that was in the mixture of solids and liquids produced by die hydrolysis reaction.

Prior to undertaking the separation of the solids from the liquids in the mixture of solids and liquids produced by the hydrolysis reaction, the mixture can be combined with, for example, the first juice obtained from pressing the feedstock. The amount of such first juice combined with the mixture of solids and liquids produced by the hydrolysis reaction can be an amount to assist with the separation of the liquid portion from the solid portion of the mixture of solids and liquids produced by the hydrolysis reaction and so that the liquid portion that is separated contains the desired amounts of water soluble sugars. Stated in another way, the amount of first juice that is combined with the mixture of solids and liquids produced by the hydrolysis reaction can be an amount so that when the separation of the solids form the liquid portion is undertaken, the soluble sugars are effectively separated from the mixture and present in the liquid portion that is separated. The liquid portion that is recovered from the separation of the mixture of solids and liquids produced by the hydrolysis reaction, either with or without the prior combination of the mixture with the first juice is hereinafter referred to as the hydrolysate.

At this stage in the described embodiment of the invention, there is the hydrolysate as described above comprising water, and water soluble sugars such as one or more or sucrose, glucose, fructose, and xylose, and there is the solids portion that was separated form the hydrolysate. The solids portion comprises cellulose that was not hydrolyzed in the hydrolysis reaction and lignin.

In certain embodiments, the starting hydrolysate solution comprises (a) total fermentable sugars at a concentration range of about 30 g/L to about 160 g/L, about 40 g/L to about 95 g/L, or about 50 g/L to about 70 g/L; (b) furfural at a concentration range of about 0.5 g/L to about 10 g/L, about 2.5 g/L to about 4 g/L, or about 1.5 g/L to about 5 g/L; (c) 5-HMF at a concentration range of about 0.1 g/L to about 5 g/L, about 0.5 g/L to about 2.5 g/L or about 1 g/L to about 2 g/L (d) acetic acid at a concentration range of about 2 g/L to about 17 g/L or about 11 g/L to about 16 g/L; (e) lactic acid at a concentration range of about 0 g/L to about 12 g/L or about 4 g/L to about 10 g/L; (t) additional aliphatic acids (e.g., succinic acid, formic acid, butyric acid and levulinic acid) at concentrations range of about 0 g/L to about 2.5 g/L; and/or (g) phenolics at a concentration range of about 0 g/L to about 10 g/L, about 0.5 g/L to about 5 g/L or about 1 g/L to about 3 g/L. In these embodiments, the starting hydrolysate solution will be referred to herein as “1×”.

In other embodiments, the starting hydrolysate can be more concentrated than 1×. For example, the starting hydrolysate solution can be about 1.5-fold, 2-fold, 3-fold, 4-fold, 5-fold, 6-fold, 7-fold, 8-fold, 9-fold or 10-fold more concentrated than 1×. In these embodiments, the starting hydrolysate will be referred to as about 1.5×, 2×, 3×, 4×, 5×, 6×, 7×, 8×, 9× and 10×, respectively.

In other embodiments, the starting hydrolysate can be less concentrated than 1×. For example, the starting hydrolysate solution can be about 0.1-fold, 0.2-fold, 0.3-fold, 0.4-fold, 0.5-fold, 0.6-fold, 0.7-fold, 0.8-fold or 0.9-fold as concentrated as 1×. In these embodiments, the starting hydrolysate will be referred to as about 0.1×, 0.2×, 0.3×, 0.4×, 0.5×, 0.6×, 0.7×, 0.8×, and 0.9, respectively.

The concentration of the hydrolysate solution can be adjusted prior to a subsequent detoxification process. Concentration of the hydrolysate solution can be particularly advantageous in the context of a continuous process. For example, a hydrolysate solution leaving a hydrolyzer following dilute acidic hydrolysis and solid/liquid separation can be concentrated prior to the addition of the magnesium base used for detoxification. In certain embodiments, the hydrolysate solution can be concentrated by about 1.5×, 2×, 3× or 5× prior to detoxification. In specific embodiments, the starting hydrolysate can concentrated in a range bounded by any two of the foregoing embodiments, e.g., concentrated by about 1× to about 3×, about 1.5× to about 3×, about 3× to about 5×, etc.

Concentrating the hydrolysate solution prior to detoxification can result in increased selectivity for furan aldehyde elimination over sugar degradation. It is believed that the rate of reaction is first order with respect to sugar degradation and second order with respect to furan aldehyde elimination. Accordingly, concentrating the hydrolysate solution results in increasing the rate of elimination of furan aldehydes relative to the rate of degradation of fermentable sugars.

The hydrolysate solution can be concentrated under reduced pressure and/or by applying heat. In one embodiment, the hydrolysate solution is concentrated in a multi-stage evaporation unit. Concentration of hydrolysate can also be performed by other technologies such as membrane filtration, carbon treatment and ion exchange resin. Evaporation results in increased sugar concentration and can result in the removal of some amounts of furfural and acetate.

In one embodiment, liquid/solid separation occurs subsequent to hydrolysis and steam-explosion, as shown in FIGS. 1A and 1B. Recovered liquid fraction is collected and fed into the detoxification vessel or reactor 50, while the remaining solids fraction, containing predominantly cellulose and lignin, are conveyed into the cake slurry tank 80.

Hydrolyzed material exits the pretreatment reactor or hydrolyzer via a discharger. The discharger can be equipped with a multiple orifice/blow-line arrangement or a valving system to steam explode material at about 25% to about 35% consistency. The steam exploded material enters blow cyclone 30, where flash steam containing concentrated furans is separated from the hydrolyzed material. The flash is used downstream in distillation to service the stripper. The blow cyclone 30 will operate at about 0 psig to 20 psig pressure, with a preferred range of about 5 to about 10 psig.

In one embodiment, the hydrolyzed material then enters an agitated tank where it is diluted with recirculated hydrolysate and potentially additional dilution consisting of juice from the front end roll mill system. Hydrolyzed material is diluted to a consistency of about 8 to about 12% with target of about 10%.

In one embodiment, the 10% slurry is then fed to three 1.4 m diameter screw presses operating in parallel showing generally as 40 in FIG. 1A. Cake will discharge from the presses at a target of about 33% consistency to the slurry tank 80. Press filtrate will discharge to the filtrate tank for transfer to detoxification in vessel or reactor 50. Press filtrate (hydrolysate) will contain approximately about 2000 to about 8000 ppm suspended solids (fines) with an anticipated target of about 5000 ppm.

### Detoxification

The hydrolysis reaction can produce one or more chemical compounds that can inhibit the conversion of the soluble sugars in the second juice to an alcohol, such as ethanol, by a fermentation process. Consequently, it is desirable to reduce the amount of or suitably eliminate these detrimental compounds in the hydrolysate prior to subjecting the hydrolysate to a process, such as a fermentation process, to convert the sugars contained therein to an alcohol such as ethanol. The one or more processes used to reduce or eliminate these detrimental compounds are referred to herein as detoxification processes. The detrimental compounds can, for example, be an aldehyde such as furfural or 5-hydroxymethyl furfural, on or more aliphatic acids, one or more esters and one or more phenolic compounds.

Various methods of detoxification have been tested, with alkaline overliming being efficient and cost effective. During the overlimning process, the pH of the hydrolysate is temporarily raised, usually at an elevated temperature, from a pH of, for example, about 2 to a pH of, for example, about 9 to about 10 through the addition of an appropriate amount of calcium hydroxide (lime). After some time, typically about 30 minutes, the pH of the hydrolysate solution is lowered through the addition of acid to a pH suitable for fermenting microorganisms. In the detoxification process, furan aldehydes are degraded and acids, both mineral and organic, are neutralized.

Overliming has been known for a long time (Leonard and Hajny, 1945, Ind. Eng. Chem., 37 (4):390-395) and still is considered an efficient detoxification method. However, a significant drawback of the method is the considerable amount of loss of fermentable sugars that occurs during detoxification. See, e.g., Larsson et al., 1999, Appl. Biochem. Biotechnol. 77-79:91-103. The loss of fermentable sugars results in lower overall yields of fermentation products such as fuels and chemicals. In addition, the formation of insoluble calcium sulfate (gypsum) during detoxification is problematic. See, e.g., Martinez et al., 2001, Biotechnol. Prog. 17(2):287-293. Gypsum formation may cause fouling and pipeline clogging, which significantly drive up maintenance costs.

As used herein, the term “detoxification” refers to a process in which one or more compounds that are detrimental to a fermenting microorganism, referred to herein as “toxins,” are removed from a starting lignocellulosic hydrolysate or inactivated, thereby forming a detoxified hydrolysate. As used herein, the phrase “detoxified hydrolysate” refers to a hydrolysate containing lower toxin levels than the toxin levels in the hydrolysate prior to the treatment in a detoxification process, referred to herein as a “starting hydrolysate”. Such toxins include, but are not limited to, furan aldehydes, aliphatic acids, esters and phenolics.

Accordingly, detoxification reduces the toxicity of a lignocellulosic hydrolysate towards a fermenting organism. An improved method involves mixing the starting hydrolysate solution, with a magnesium base such as one or more of magnesium hydroxide, magnesium carbonate or magnesium oxide, for a period of time and under conditions that result in the production of a solution of a detoxified hydrolysate without formation of gypsum. This method results in a detoxified hydrolysate in which the quantity of the toxins that are deleterious to fermenting microorganisms is substantially reduced relative to the starting hydrolysate. At the same time, the amount of fermentable sugars lost in the detoxification process is suitably minimized.

In certain embodiments, the methods disclosed herein result in the production of a detoxified hydrolysate with at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% of the fermentable sugars present in the starting hydrolysate and no greater than about 70%, no greater than about 60%, no greater than about 50%, no greater than about 40%, no greater than about 30%, no greater than about 20% or no greater than about 10% of the furan aldehydes present in the staring hydrolysate. In particular embodiments, detoxification methods of the present disclosure provide a detoxified hydrolysate with (a) at least about 90% of the total fermentable sugars present in the starting hydrolysate and no greater than about 50% of the furan aldehydes present in the starting hydrolysate; (b) at least about 90% of the total fermentable sugars present in the starting hydrolysate and no greater than about 40% of the furan aldehydes present in the starting hydrolysate; (c) at least about 90% of the total fermentable sugars present in the starting hydrolysate and no greater than about 30% of the furan aldehydes present in the starting hydrolysate; (d) at least about 90% of the total fermentable sugars present in the starting hydrolysate and no greater than about 20% of the furan aldehydes present in the starting hydrolysate; (e) at least about 80% of the total fermentable sugars present in the starting hydrolysate and no greater than about 50% of the furan aldehydes present in the starting hydrolysate; (f) at least about 80% of the total fermentable sugars present in the starting hydrolysate and no greater than about 40% of the furan aldehydes present in the starting hydrolysate; (g) at least about 80% of the total fermentable sugars present in the starting hydrolysate and no greater than about 30% of the furan aldehydes present in the starting hydrolysate; or (h) at least about 80% of the total fermentable sugars present in the starting hydrolysate and no greater than about 20% of the furan aldehydes present in the starting hydrolysate.

The concentration of the individual compounds of the hydrolysate in the hydrolysate solution prior to detoxification depends, in part, on the feedstock from which the hydrolysate is obtained and the method used to hydrolyze the feedstock, as well as hydrolysis conditions. In certain embodiments, the starting hydrolysate solution comprises (a) fermentable sugars at a concentration range of about 30 g/L to about 160 g/L, about 40 g/L to about 95 g/L, or about 50 g/L to about 70 g/L; (b) furfural at a concentration range of about 0.5 g/L to about 10 g/L, about 2.5 g/L to about 4 g/L, or about 1.5 g/L to about 5 g/L; (c) 5-HMF at a concentration range of about 0.1 g/l to about 5 g/L, about 0.5 g/L to about 2.5 g/L or about 1 g/L to about 2 g/L; (d) acetic acid at a concentration range of about 2 g/L to about 17 g/L or about 11 g/L to about 16 g/L; (e) lactic acid at a concentration range of about 0 g/L to about 12 g/L or about 4 g/L to about 10 g/L; (f) additional aliphatic acids (e.g., succinic acid, formic acid, butyric acid and levulinic acid) at a concentration range of about 0 g/L to about 2.5 g/L; and/or (g) phenolics at a concentration range of about 0 g/L, to about 10 g/L, about 0.5 g/L to about 5 g/L or about 1 g/L to about 3 g/L.

The starting hydrolysate solution can be concentrated prior to detoxification. For instance, following feedstock hydrolysis, a hydrolysate solution can be concentrated by about 1.2-fold, 1.3-fold, 1.4-fold, 1.5-fold, 1.6-fold, 1.7-fold, 1.8-fold, 1.9-fold, 2-fold, 3-fold, 4-fold, or 5-fold. In specific embodiments, the starting hydrolysate is concentrated in a range bounded by any two of the foregoing embodiments, e.g., concentrated in the range of about 1-fold to about 3-fold, about 1.5-fold to about 3-fold, about 3-fold to about 5-fold, etc.

In various embodiments, the detoxification of the lignocellulosic hydrolysate solution can be carried out at a temperature range of about 25° C. to about 90° C. The detoxification process can be carried out, for example at about 30° C., 35° C., 40° C., 45° C., 50° C., 55° C., 60° C., 65° C., 70° C., 75° C., 80° C., 85° C., or 90° C. In specific embodiments, the detoxification process is carried out at a temperature in the range bounded by any two of the foregoing temperatures, e.g., at a temperature range of about 40° C. to about 60° C., about 45° C. to about 50° C., about 35° C. to about 65° C., etc. Advantageously, the detoxification process is carried out at a temperature range of about 40° C. to about 60° C., which allows the detoxification reactions to occur at a commercially feasible rate while minimizing the loss of fermentable sugars, and thereby increasing the yield of fermentation products (e.g., ethanol, biochemicals).

The hydrolysate detoxification process is typically carried out at a pH range of about 6.2 to about 9.5. For instance, the detoxification can be carried out at a pH of about 6.5, 7, 7.5, 8, 8.5, 9, or 9.5. In specific embodiments, the detoxification process can be carried out at a pH in the range bounded by any two of the foregoing values, e.g., at a pH range of about 6.5 to about 8, about 7 to about 8, etc.

In certain aspects, the disclosure provides for a method of reducing the toxicity of a lignocellulosic hydrolysate towards a fermenting organism, comprising the step of mixing a starting lignocellulosic hydrolysate solution, said starting lignocellulosic hydrolysate solution comprising a mixture of fermentable sugars, furan aldehydes and aliphatic acids, with a magnesium base for a period of time of at least about 1 hour, at least about 4 hours, at least about 10 hours or at least about 20 hours at a temperature of about 40° C. to about 70° C. and at a pH of about 6.5 to about 8. In particular embodiments, the magnesium base is magnesium hydroxide.

The detoxification process can be carried out as a batch process, as a continuous process, or as a semi-continuous process. The detoxification process can be carried out in a batch reactor, a continuous stirred tank reactor (CSTR), a series of continuous stirred tank reactors, or a plug flow reactor (PFR).

The disclosure further provides methods for continuously reducing the quantity of toxins in a hydrolysate, comprising the steps of flowing a first continuous stream of a hydrolysate into a continuous reactor or a series of continuous reactors, flowing a second continuous stream of a solution of a magnesium base into the continuous reactor or the series of continuous reactors, mixing the hydrolysate with the magnesium base in the continuous reactor for a period of time sufficient to reduce the quantity of toxins in the hydrolysate, and flowing the hydrolysate out of the continuous reactor.

Suitable detoxification processes are described, for example, in U.S. Provisional Patent Application Ser. No. 61/597,936, filed Feb. 13, 2012, and U.S. Provisional Patent Application Ser. No. 61/597,973, filed Feb. 13, 2012. One suitable process comprises temporarily increasing the pH of the hydrolysate, suitable while at an elevated temperature, to a pH of about 9 to about 10 by combining calcium hydroxide with the hydrolysate. After a suitable amount of time, for example, about 30 minutes, at this pH and, optionally, at an elevated temperature, the pH of the hydrolysate is lowered by, for example, the addition of a suitable acid such as sulfuric acid, to a pH that is acceptable for fermentation of the sugars in the detoxified hydrolysate to fermentation products such as ethanol. Another suitable process comprises increasing the pH of the hydrolysate to about 5 or to about 6 using, for example, one or more basic compounds, such as one or more of ammonium hydroxide, sodium hydroxide, potassium hydroxide, calcium hydroxide, magnesium hydroxide, magnesium carbonate or magnesium oxide. The aforementioned magnesium-containing bases are advantageous. The amount of base that is used is an amount that achieves the desired pH. The temperature for the first step can be about 25° C. or greater and can be up to, for example, about 90° C. The detoxification process can be carried out for about 15 minutes to about 40 hours.

It is also possible to use a detoxification procedure that comprises two steps using a first base for the first step and a second base for the second step. The first step of the detoxification process can comprise combining the hydrolysate with a first base or first mixture of bases at a pH of, for example, about 3 to about 9, for example at a pH of about 3 to about 4, about 3 to about 5, or about 4 to about 6. In the second step the hydrolysate after treatment in the first step is combined with a second base or second mixture of bases at a pH ranging from about 7 to about 10, for example at a pH of about 7, 8, 9 or 10. In specific embodiments, the pH is in the range bounded by any of the two foregoing embodiments, e.g., a pH range of about 7 to about 9, about 8 to about 9, about 8 to about 10. The first base can be any suitable base and can be, for example, one or more of magnesium hydroxide, magnesium carbonate or magnesium oxide. The second base can be the same as the first base but can also be, for example, one or more of ammonium hydroxide, ammonia, sodium hydroxide, potassium hydroxide, calcium hydroxide. The amount of base that is used is an amount that achieves the desired pH. The temperature for the first step can be about 25° C. or greater and can be up to, for example, about 90° C. The first step of the two step detoxification process can be carried out for about 1 minute to about 15 minutes and the second step for about 30 minutes to about 20 hours.

Detoxification of Hydrolysates with a Base

The detoxification methods of the disclosure generally entail mixing a lignocellulosic hydrolysate with a base such as, for example but not limited thereto, sodium hydroxide, sodium bicarbonate, potassium hydroxide, magnesium hydroxide, barium hydroxide, aluminum hydroxide, ferrous hydroxide, ferric hydroxide, zince hydroxide, lithium hydroxide, calcium hydroxide, ammonium hydroxide, ammonia, or a magnesium base (e.g., magnesium hydroxide, magnesium carbonate or magnesium oxide) for a period of time and under conditions that result in the production of a detoxified lignocellulosic hydrolysate.

The detoxification methods are highly selective towards elimination of furan aldehydes. As used herein, the phrase “highly selective towards elimination of furan aldehydes” refers to the observation that furan aldehydes react with magnesium bases at higher rates than fermentable sugars react with magnesium bases. As a result, the detoxified hydrolysates produced in accordance with the present disclosure have a larger percentage of fermentable sugars and a lower percentage of furan aldehydes relative to the starting hydrolysate. The detoxified hydrolysates can then be fermented by a suitable fermenting microorganism to produce a fermentation product such as ethanol.

The detoxification methods typically comprise mixing a starting lignocellulosic hydrolysate solution with a magnesium base for a period of time and under conditions that result in the production of a detoxified hydrolysate solution. The amount of time suitable to perform the detoxification process depends on a number of factors, including the chemical composition of the hydrolysate, the concentration of the hydrolysate solution, the reaction temperature, the pH of the hydrolysate solution, the total amount of magnesium base added, the stirring rate, and the type of reactor being used. The detoxification process is typically carried out for a period of time in the range of about 15 minutes to about 80 hours, and more typically in the range of about 1 hour to about 40 hours. In specific embodiments, the detoxification process is carried out for a period of time in the range of about 1 hour to about 30 hours, about 1.5 hours to about 20 hours. about 2 hours to about 12 hours, about 3 hours to about 9 hours, about 4 hours to about 10 hours, or about 6 hours to about 9 hours. This process is applicable for batch and continuous vessel treatments.

The hydrolysate detoxification process is typically carried out at a temperature of 90° C. or less, for example at a temperature of about 30° C., 35° C., 40° C., 45° C., 50° C., 55° C., 60° C., 70° C., 75° C., 80° C. or 85° C. In specific embodiments, the temperature is in the range bounded by any of the two foregoing embodiments, such as, but not limited to, a temperature range of about 40° C. to about 70° C., about 40° C. to about 60° C., about 40° C. to about 55° C., about 45° C. to about 55° C., about 45° C. to about 50° C., about 50° C. to about 55° C., or about 40° C. to about 50° C. In particular embodiments, the temperature of the hydrolysate solution is in the range of about 40° C. to about 60° C. In this range, high selectivity for furan aldehyde elimination is achieved while commercially feasible rates of the detoxification reactions are observed.

The hydrolysate detoxification process is typically carried out at a pH range of about 6.2 to about 9.5, for example at a pH of about 6.5, 7, 7.5, 8, 8.5, 9.0 or 9.5. In specific embodiments, the pH is in the range bounded by any of the two foregoing values, such as, but not limited to, a pH range of about 6.5 to about 8, about 6.5 to about 7.5, about 7 to about 8, or about 7 to about 7.5. It will be understood that the pH of the hydrolysate solution depends on the concentration of the magnesium base and the temperature of the solution. In embodiments where hydrolysate detoxification is carried out using magnesium hydroxide, the solubility of the magnesium hydroxide decreases with increasing temperature. Therefore, for a given amount of magnesium hydroxide added to the hydrolysate solution, the equilibrium pH decreases as the temperature is increased, all other variables being constant. The pH of the solution can decrease slightly as the detoxification process progresses owing to the consumption of hydroxide in reaction with sugars and furans. Additional magnesium hydroxide can be added to the hydrolysate solution to adjust the pH during the course of the reaction.

The total amount of magnesium base added to hydrolysate solution 1× can be in the range of about 2 grams per 1 kilogram hydrolysate (2 g/kg hydrolysate) to about 200 grams per 1 kilogram hydrolysate (200 g/kg hydrolysate). For instance, the total amount of magnesium base added to the hydrolysate solution can be about 40 g/kg hydrolysate, about 80 g/kg hydrolysate, about 100 g/kg hydrolysate, about 120 g/kg hydrolysate, about 140 g/kg hydrolysate, or about 160 g/kg hydrolysate. The magnesium base can be added to the hydrolysate solution in a single step, in multiple portions or continuously throughout the course of the detoxification process. In specific embodiments, the total amount of magnesium base added to the hydrolysate solution is in the range bounded by any of the two foregoing embodiments, such as, but not limited to, about 40 g/kg hydrolysate to about 160 g/kg hydrolysate, about 40 g/kg hydrolysate to about 120 g/kg hydrolysate, about 80 g/kg hydrolysate to about 160 g/kg hydrolysate, about 80 g/kg hydrolysate to about 140 g/kg hydrolysate, or about 140 g/kg hydrolysate to about 160 g/kg hydrolysate. For more concentrated hydrolysate solutions (e.g., 4×), the amount of magnesium base sufficient to raise the pH to the desired level would be increased relative to hydrolysate solution 1×. For less concentrated hydrolysate solutions (e.g., 0.5×), the amount of magnesium base sufficient to raise the pH to the desired level would be decreased relative to hydrolysate solution 1×.

The hydrolysate detoxification process can be performed in any suitable vessel, such as a batch reactor or a CSTR or a PFR. A CSTR allows for continuous addition and removal of input materials (e.g., hydrolysate, magnesium base slurry) as the detoxification reaction progresses. The suitable vessel can be equipped with a means, such as impellers, for agitating the hydrolysate solution. Reactor design is discussed in Lin, K.-H., and Van Ness, H. C. (in Perry, R. H. and Chilton, C. H. (eds), Chemical Engineer's Handbook, 5th Edition (1973) Chapter 4, McGraw-Hill, NY.

The detoxification processes can be carried out in a batch mode. The methods typically involve combining the hydrolysate solution and the magnesium base (or magnesium base slurry) in the reactor. The hydrolysate solution and the magnesium base can be fed to the reactor together or separately. Any type of reactor can be used for batch mode detoxification, which simply involves adding material, carrying out the detoxification process at specified conditions (e.g. temperature, dosage and time) and removing the detoxified hydrolysate from the reactor.

Alternatively, the detoxification processes can be carried out in a continuous mode. The continuous processes of the disclosure advantageously reduces the need to stop and clean reactors and accordingly can be carried out in continuous mode, e.g., for periods of several days or longer (e.g., a week or more) to support an overall continuous process. The methods typically entail continuously feeding hydrolysate solution and magnesium base slurry to a reactor. The hydrolysate and the magnesium base slurry can be fed together or separately. The resultant mixture has a particular retention or residence time in the reactor. The residence time is determined by the time to achieve the desired level of detoxification following the addition of the hydrolysate and the base to the reactor. Following the detoxification process, the detoxified hydrolysate exits the reactor and additional components (e.g., hydrolysate and base slurry) are added to the reactor. Multiple such reactors can be connected in series to support further pH adjustment during an extended retention time and/or to adjust temperature during an extended retention time

For detoxification in continuous mode, any reactor can be used that allows equal input and output rates, e.g., a continuous stirred tank reactor or plug flow reactor, so that a steady state is achieved in the reactor and the fill level of the reactor remains constant.

The detoxification processes of the disclosure can be carried out in semi-continuous mode. Semi-continuous reactors, which have unequal input and output streams that eventually require the system to be reset to the starting condition, can be used.

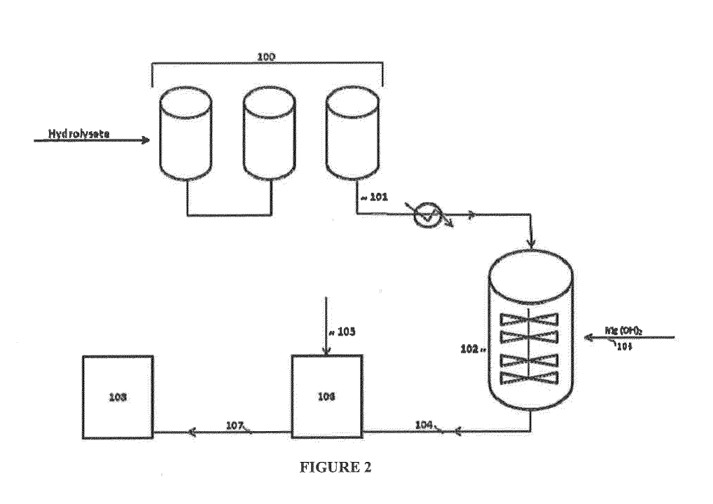

The present disclosure provides methods of continuously detoxifying a feedstock obtained from a lignocellulosic feedstock. As depicted in FIG. 2, steps of the continuous detoxification process include flowing a first continuous stream of a hydrolysate into a continuous reactor, flowing a second continuous stream of a solution of a magnesium base into the continuous reactor, mixing the hydrolysate with the magnesium base in the continuous reactor for a period of time sufficient to reduce the quantity of toxins in the hydrolysate, and flowing the hydrolysate out of the continuous reactor.

The methods of the disclosure can include further steps in addition to detoxification, such as one or more steps depicted in FIG. 2 that are upstream or downstream of the detoxification step. In FIG. 2, only steps that are downstream of feedstock hydrolysis are depicted. Following hydrolysis of the feedstock and solid/liquid separation, the hydrolysate is concentrated in a multi-stage evaporation unit 100. The hydrolysate leaves the multi-stage evaporation unit 100 through line 101 and enters continuous stirred tank reactor 102. The hydrolysate can be heated prior to entering continuous stirred tank reactor 102. Magnesium hydroxide is supplied continuously to the continuous stirred tank reactor through line 103. After the detoxificatioil in continuous stirred tank reactor 102 is complete, the detoxified hydrolysate is passed into line 104, where it is met with a stream of acid (e.g., sulfuric acid or phosphoric acid) from line 105. The mixture of detoxified hydrolysate is passed into mixer 106. The neutralized detoxified hydrolysate exits mixer 106 through line 107 and flows into fermentation vessel 108.

Adequate mixing of the hydrolysate solution following addition of the magnesium base can improve the rate of dissolution of the base and ensure that the pH remains substantially homogeneous throughout the solution. For instance, ideal mixing will avoid the formation of local pockets of higher pH, which can result in lower selectivity for furan elimination. Mixing speeds of about 100 revolutions per minute (rpm) to about 1500 rpm can be used to ensure sufficient mixing of the hydrolysate solution. For instance, mixing speeds of about 100 rpm, 200 rpm, 300 rpm, 400 rpm, 500 rpm, 600 rpm, 700 rpm, 800 rpm, 900 rpm, 1000 rpm, 1100 rpm, 1200 rpm, 1300 rpm, 1400 rpm, and 1500 rpm can be used. In specific embodiments, mixing is carried out at speeds bounded by any two of the foregoing mixing speeds, such as, but not limited to about 100 rpm to about 200 rpm, about 100 rpm to about 400 rpm, about 200 rpm to about 400 rpm, about 400 rpm to about 800 rpm or about 800 rpm to about 1,500 rpm. In other embodiments, intermittent mixing regimes can be used where the rate of mixing is varied as the detoxification process progresses. Mixing of the hydrolysate solution can be accomplished using any mixer known in the art, such as a high-shear mixer, paddle mixer, magnetic stirrer or shaker, vortex, agitation with beads, and overhead stirring.

The detoxification methods of the present disclosure provide detoxified hydrolysates in which a substantial portion of the furan aldehydes have been removed relative to the starting hydrolysate. At the same time, the detoxification results in minimal loss of fermentable sugars. Therefore, the detoxification reactions are highly selective towards elimination of furan aldehydes. In particular embodiments, methods disclosed herein provide a detoxified hydrolysate with at least about 70%, at least about 80%, at least about 85%, at least about 90%, at least about 95% or at least about 99% of the fermentable sugars present in the starting hydrolysate and no greater than about 50%, no greater than about 40%, no greater than about 30%, or no greater than about 20% of the furan aldehydes present in the staring hydrolysate solution.

In particular embodiments, detoxification methods of the present disclosure provide a detoxified hydrolysate with (a) at least about 90% of the total fermentable sugars present in the starting hydrolysate and no greater than about 50% of the furan aldehyde present in the starting hydrolysate; (b) at least about 90% of the total fermentable sugars present in the starting hydrolysate and no greater than about 40% of the furan aldehydes present in the starting hydrolysate; (c) at least about 90% of the total fermentable sugars present in the starting hydrolysate and no greater than about 30% of the furan aldehydes present in the starting hydrolysate; (d) at least about 90% of the total fermentable sugars present in the starting hydrolysate and no greater than about 20% of the furan aldehydes present in the starting hydrolysate; (e) at least about 80% of the total fermentable sugars present in the starting hydrolysate and no greater than about 50% of the furan aldehydes present in the starting hydrolysate; (f) at least about 80% of the total fermentable sugars present in the starting hydrolysate and no greater than about 40% of the furan aldehydes present in the starting hydrolysate; (g) at least about 80% of the total fermentable sugars present in the starting hydrolysate and no greater than about 30% of the furan aldehydes present in the starting hydrolysate; (h) at least about 80% of the total fermentable sugars present in the starting hydrolysate and no greater than about 20% of the furan aldehydes present in the starting hydrolysate.

In one embodiment, the liquid fraction contains a mixture of sugars resulting from the acid hydrolysis of hemicellulose, glucose from the acid hydrolysis of some cellulose, acetate from acetyl groups, soluble lignin and other components. The pH of this stream leaving liquid solid separation will be in the range of about 1.5 to about 2.5. This stream will be either detoxified by the addition on an insoluble base such as magnesium hydroxide or magnesium oxide then with ammonium hydroxide to a pH greater than about 8.0. Alternatively, ammonium hydroxide will be added to the liquid stream to a pH in the range of about 5.5 to about 6.8 depending on the tolerance of the ethanologen yeast to the liquid stream toxicity.

Hydrolysate containing soluble sugars (sucrose, fructose, glucose, xylose) recovered after hydrolysis and liquid-solid separation is fed into detoxification in order to remove inhibitory compounds (eg. furfural, 5-hydroxymethyl furfural).

Detoxification occurs by first raising the pH of the mixed-juice hydrolysate starting at pH in the range of about 3 to about 4 to a pH in the range of about 5 to about 6. This pH neutralization step is accomplished by addition of magnesium hydroxide in a single stage vessel at a temperature of about 45° C. The residence time for pH neutralization is approximately about 30 minutes.

After neutralization with magnesium hydroxide, pH is further adjusted using ammonia in three vessels in series. Ammonia is added into the first reaction vessel to a pH of about 8.3 to about 8.8 and about 55° C. Adjusted hydrolysate is transferred in sequence to the next two reaction vessels for a total residence time of about 180 minutes.

During the sequential pH adjustment steps, toxic aldehydes are converted into non-toxic compounds. Also, up to about 5% of the available sugar is converted into unfermentable compounds during detoxification. Optionally, the pH of the detoxified material is reduced again to a pH of about 6 to about 7 with the addition of phosphoric acid. Fully detoxified, neutralized hydrolysate is then sent to primary fermentation.

### Fermentation Organism

Although a mixture of fermentation microorganisms, such as one or more different kinds of yeasts, can be used, suitably, a single fermentation organism such as a single kind of yeast that is capable of fermenting both pentose and hexose sugars is used in embodiments of this invention. The microorganism can be a wild type of microorganism or a recombinant microorganism, and can include, for example, Escherichia, Zymomonas, Saccharomyces, Candida, Pichia, Streptomyces, Bacillus, Schizosaccharomyces, Dekkera, Bretanomyces, Kluyveromyces, Issatchenkia, Hansenula, Pachysolen, Torulaspora, Zygosaccharomyces, Yarrowia, Lactobacillus, and Clostridium. Particularly suitable species of fermenting microorganisms include Escherichia coli, Z. mobilis, Bacillus stearothermophilus, Clostridia thermocellum, and Thermoanaerobacterium saccharolyticum. Genetically modified strains of E. coli or Zymomonas mobilis can be used for ethanol production (see, e.g., Underwood et al., 2002, Appl. Environ. Microbiol. 68:6263-6272 and US 2003/0162271 A1).

More specific examples of suitable fermentation organisms include, for example, S. cerevisiae, S. carlsbergensis, S. pastorianus, BioTork strain SC48-EVG51, Schizosaccharomyces pombe, D. bruxellensis, D. Anomala, B. bruxellensis, B. anomalus, B. cuslerianus, B. naardensis, B. nanus, K. marxianus. K. lactis, C. sonorensis, C. methanosorbosa, C. ethanolica, C. maltose, C. tropicalis, C. albicans, C. stellate, C. shehatae, I. orientalis (also known as Pichia kudriavzevii and the anamorph form (asexual form) known as Candida krusei), ATCC 3196, ATCC PTA-6658, Issatchenkia kudryavtsev, Cargill strain 1822, Cargill strain 3556, Cargill strain 3085, Cargill strain 3849, Cargill strain 3859, H. polymorpha ML3, H. polymorpha ML9, H. polymorpha ML6, H. polyymorpha ML8, H. polymorpha N95, P. tannophilus. P. tannophilus strain NRRL 2460, P. tannophilus strain I fGB 0101, P. stipitis (now known as Scheffersomyces stipitis), Scheffersomyces stipitis strain CBS 6054, Scheffersomyces stipitis NRRL 7124, Scheffersomyces stipitis NRRL 11545, P. fermentans, P. faleiformis. P. sp. YB-4149, P. deserticola, P. membranifaciens, P. galeibormis, P. segobiensis, P. segobiensis strain NRRL 11571, T. delbruekii, Z. bailii, and Y. lipolytica.

The microorganism can be propagated in one or more separate vessels shown as 70 in FIGS. 1A and 1B located at or near the vessels used to undertake the fermentation steps in the embodiments of this invention. The microorganism selected for the fermentation can be propagated in one or more suitable, hygienic vessels, at a pH of about 5.8 to about 6.2, at a temperature at about 30° C. to about 35° C., or about 32° C. to about 35° C. Aeration and agitation can be used to assist with the propagation. The specific growth rate of the microorganism can be about 0.12 to about 0.18 hr⁻¹, or about 0.12 to about 0.15 hr⁻¹. For example, the microorganism can be propagated in a series of successively larger vessels by extracting a portion of the contents of a vessel and using it to inoculate the contents of a larger vessel. In that way, a large supply of the microorganism can be prepared. For example, inoculate from vessels ranging about 10 to about 30 gallons can be used to inoculate the contents of vessels of about 400 to about 600 gallons, and inoculate from these vessels can be used to inoculate the contents of vessels of about 10,000 to about 15,000 gallons, and inoculate from these vessels can be used to inoculate the contents of vessels of about 20,000 to about 30,000 or 40,000 gallons. The yeast concentration in the vessels can be, for example, about 1×10⁶ CFU/mL to about 1×10⁸ CFU/mL.

In one embodiment, the fermentation organism is a single yeast capable of fermenting both pentose and hexose sugars such as S. cerevisiae. This yeast will be propagated onsite and fed into the primary fermentation vessel 60. Yeast added to the primary fermentation vessel will then move forward into the secondary fermentation vessel 90 where they will also convert sugar liberated from the pretreated cake to ethanol.

The use of yeast is well known technology, with well developed process steps to propagate, feed, and store in a hygienic manner to a fermentor. Generally, the yeast will be propagated at a pH of about 5.5, and temperature of about 35° C., with aeration in order to achieve yeast specific growth rate of about 0.12 h⁻¹. Actively growing yeast cultures will be inoculated into successively larger vessels of incrementally increasing volume of about 21 gallons, about 425 gallons, about 12,500 gallons, and final stage of about 25,000 gallons. Yeast will be used to inoculate the primary fermentation when a yeast concentration of about 5×10⁷ CFU/mL is achieved. Yeast will be inoculated into the primary fermentation vessel 60 at a maximum pitching rate of about 2 gDW/L after inoculation.

A primary fermentor will be charged with the yeast ethanologen which has been propagated through a separate propagation train. The propagation train will begin with a cell bank vial containing about 1.5 mL of the yeast that will be amplified through successive passages from by adding the about 1.5 mL to about 100 to about 200 mL of growth media in a shake flask. This will be allowed to incubate at about 30 to about 35° C. for about 24 to about 36 hours until high levels of yeast growth is achieved and the contents of the flask will be transferred to a larger flask or more preferably a large sterile bag rocking fermentor of about 10 to about 20 L volume. This is again incubated at about 30 to about 35° C. for about 24 to about 36 hours until a high level of yeast growth is achieved and the contents of the bag are transferred to a fermentor with a working volume of about 1000 to about 3000 L containing a base yeast growth medium. The fermentor is to be operated at a temperature of about 30 to about 35° C. for about 24 to about 36 hours with the pH controlled by the addition of ammonium hydroxide within a range of about 5.5 to about 6.8. The fermentation will be fed with liquid glucose and will be highly aerated to ensure oxygen is not limiting. The feeding with liquid glucose will continue until a cell mass of about 20 to about 50 g/L DCW is achieved and the contents will be transferred to a fermentor with a working volume of about 10000 to about 30000 L containing a base yeast growth medium. The fermentor is to be operated at a temperature of about 30 to about 35° C. for about 24 to about 36 hours with the pH controlled by the addition of ammonium hydroxide within a range of about 5.5 to about 6.8. The fermentation will be fed with liquid glucose and will be highly aerated to ensure oxygen is not limiting. The feeding with liquid glucose will continue until a cell mass of about 20 to about 50 g/L DCW is achieved and the contents will be transferred to a fermentor with a working volume of about 100000 to about 200000 L containing a base yeast growth medium. The fermentor is to be operated at a temperature of about 30 to about 35° C. for about 24 to about 36 hours with the pH controlled by the addition of ammonium hydroxide within a range of about 5.5 to about 6.8. The fermentation will be fed with liquid glucose and detoxified hydrolysate and will be highly aerated to ensure oxygen is not limiting. The feeding of hydrolysate will have a target of about 20% final volume to ensure adaptation of the yeast with the hydrolysate. Feeding with the detoxified hydrolysate and liquid glucose will continue until a cell mass of about 40 to about 60 g/L DCW is achieved. A fraction of the final propagator volume will be removed about 7760 gallons (29375 L) (388,000 gallons final secondary fermentor volume (1,468,735 L) at about 1 g/L divided by about 50 g/L in final propagation stage). The feeding with the liquid glucose and hydrolysate will continue for a second through seventh withdrawal at which point the final propagation would be terminated and the propagation train would restart from the working cell bank vial.

### Saccharification Enzymes

Hydrolyzing proteins suitable for saccharification of the pretreated feedstock include cellulases, hemicellulases (including but not limited to xylanases, mannanases, beta-xylosidases), and other proteins that enhance saccharification by cellulase or hemicellulases, such carbohydrate esterases (including but not limited to acetyl xylan esterases and ferulic acid esterases), laccases (which are believed to act on lignin), and non-enzymatic proteins such as swollenins (which are thought to swell the cellulose (non-catalytically and make it more accessible to cellulases). As used herein, the term hydrolyzing proteins refers to a single protein, preferably an enzyme (yet more preferably a cellulase or hemicellulase) or a cocktail of different proteins, including one or more enzymes (preferably a cellulase and/or hemicellulase) and optionally one or more non-enzymatic proteins such as swollenins. The hydrolyzing proteins can have naturally occurring or engineered polypeptide sequences.

Biomass typically contains cellulose, which is hydrolyzable into glucose, cellobiose, and higher glucose polymers and includes dimers and oligomers. Cellulose is hydrolysed into glucose by the carbohydrolytic cellulases. Thus the carbohydrolytic cellulases are examples of catalysts for the hydrolysis of cellulose. The prevalent understanding of the cellulolytic system divides the cellulases into three classes; exo-1,4-β-D-glucanases or cellobiohydrolases (CBH) (EC 3.2.1.91), which cleave off cellobiose units from the ends of cellulose chains; endo-1,4-β-D-glucanases (EG) (EC 3.2.1.4), which hydrolyse internal-1,4-glucosidic bonds randomly in the cellulose chain; 1,4-β-D-glucosidase (EC 3.2.1.21), which hydrolyses cellobiose to glucose and also cleaves off glucose units from cellooligosaccharides. Therefore, if the biomass contains cellulose, suitable hydrolyzing enzymes include one or more cellulases.

Many biomasses include hemicellulose, which is hydrolyzable into xylan, glucuronoxylan, arabinoxylan, glucomannan, and xyloglucan. The different sugars in hemicellulose are liberated by the hemicellulases. The hemicellulytic system is more complex than the cellulolytic system due to the heterologous nature of hemicellulose. The systems may involve among others, endo-1,4-β-D-xylanases (EC 3.2.1.8), which hydrolyze internal bonds in the xylan chain; 1,4-β-D-xylosidases (EC 3.2.1.37), which attack xylooligosaccharides from the non-reducing end and liberate xylose; endo-1,4-β-D-mannanases (EC 3.2.1.78), which cleave internal bonds; 1,4-β-D-mannosidases (EC 3.2.1.25), which cleave mannooligosaccharides to mannose. The side groups are removed by a number of enzymes; such as α-D-galactosidases (EC 3.2.1.22), α-L-arabinofuranosidases (EC 3.2.1.55), α-D-glucuronidases (EC 3.2.1.139), cinnamoyl esterases (EC 3.1.1.), acetyl xylan esterases (EC 3.1.1.6) and feruloyl esterases (EC 3.1.1.73). Therefore, if the biomass contains hemicellulose, suitable hydrolyzing enzymes include one or more hemicellulases.

The cellulase cocktails suitable for saccharification of the pretreated feedstock include one or more cellobiohydrolases, endoglucanases and/or β-glucosidases. Cellulase cocktails are compositions comprising two or more cellulases. In their crudest form, cellulase cocktails contain the microorganism culture that produced the enzyme components. “Cellulase cocktails” also refers to a crude fermentation product of the microorganisms. A crude fermentation is preferably a fermentation broth that has been separated from the microorganism cells and/or cellular debris (e.g., by centrifugation and/or filtration). In some cases, the enzymes in the broth can be optionally diluted, concentrated, partially purified or purified and/or dried.

Suitable cellulases include those of bacterial or fungal origin. Suitable cellulases include cellulases from the genera Bacillus, Pseudomonas, Trichoderma, Aspergillus, Chrysosporiuim, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259. The Trichoderma reesei cellulases are disclosed in U.S. Pat. No. 4,689,297, U.S. Pat. No. 5,814,501, U.S. Pat. No. 5,324,649, WO 92/06221 and WO 92/06165. Bacillus cellulases are disclosed in U.S. Pat. No. 6,562,612.

Commercially available cellulases or cellulase cocktails that can suitably be used in the present methods include, for example, CELLIC CTec (Novozymes), ACCELLERASE (Genencor), SPEZYME CP (Genencor), 22 CG (Novozymes), Biocellulase W (Kerry) and Pyrolase (Verenium), Novozyme-188 β-glucosidase (Novozymes), AlternaFuel® CMAX™ (Dyadic), AlternaFuel® 100P (Dyadic), AlternaFuel® 200P (Dyadic), AlternaFuel® CMAX3™ (Dyadic), Cellic CTec3 (Novozymes), Cellic CTec2 (Novozymes), Cellic CTec (Novozymes), Cellic HTec3 (Novozymes), Accellerase® TRIO (Genencor).

In some embodiments, the cellulase cocktail includes one or more proteins not normally produced by the cellulase-producing microorganism. The non-native proteins can be foreign or engineered proteins recombinantly co-expressed with other cellulase cocktail components by a cellulase-producing microorganism (e.g., bacterium or fungus). or natively or recombinantly produced separately from other cellulase components (e.g., in a bacterium, plant or fungus) and added to the cellulase cocktail. Mixtures of enzymes from different organisms can also be used.

Hydrolyzing proteins can be used singly or in enzyme/cocktail blends in doses in the range of about 5 μg to about 20 mg protein per gram dry weight of solids in the slurry, e.g., about 5 μg, 10 μg, 20 μg, 50 μg, 100 μg, 250 μg, 500 μg, 1 mg, 2 mg, 5 mg, 10 mg, or 20 mg protein per gram dry weight of solids in the slurry. In various embodiments, the dosage per gram dry weight of solids in the slurry is in a range bounded by any two of the foregoing embodiments, such as about 10 μg to about 250 μg, about 20 μg to about 500 μg, about 50 μg to about 250 μg, about 10 μg to about 100 μg, or about 20 μg to about 250 μg, about 100 μg to 1 about 0 mg, about 250 μg to about 20 mg, etc.

The term CTU as used herein refers to units of cellulase activity as measured using CELLAZYME T tablets (Megazyme, Co. Wickow, Ireland). The substrate in this assay is azurine-crosslinked Tamarind Xyloglucan (AZCL-Xyloglucan). This substrate is prepared by dyeing and cross-linking highly purified xyloglucan to produce a material which hydrates in water but is water insoluble. Hydrolysis by cellulase, for example, endo-(1-4)-b-D-glucanase, produces water soluble dyed fragments and the rate of release of these (increase in absorbance at about 590 nm) can be related directly to enzyme activity. One CTU is defined as the amount of enzyme required to release one micromole of glucose reducing sugar-equivalents per minute from barley β-glucan (10 mg/mL) at a pH of about 4.5 and about 40° C. 7.5 CTUs of cellulase cocktail correspond to approximately 1 filter paper unit (“FPU”). As used herein, the term FPU refers to filter paper units as determined by the method of Adney and Baker, Laboratory Analytical Procedure #006 (“IAP-006”), “Measurement of cellulase activity,” Aug. 12, 1996, the USA National Renewable Energy Laboratory (NREL), which is expressly incorporated by reference herein in entirety. A mass of about 1 mg of total protein of a T. reesei cellulase cocktail (as measured by the Bradford assay) corresponds to approximately about 27.4 CTU. In alternative embodiments of the present invention, the reference to enzyme dosages in “CTUs” can be replaced with the approximate corresponding amounts of enzyme by protein mass or FPUs, using the conversion of about 36.5 μg of a cellulase or cellulase cocktail or about 0.133 FPU of a cellulase or cellulase cocktail per CTU. Accordingly, in these alternative embodiments, enzyme dosages referred to by CTUs in the various aspects of the disclosure are substituted by the corresponding dosage in protein mass or FPU. Thus, for example, alternatives to an embodiment of saccharification methods in which the enzyme dose is about 20 to about 400 CTU are embodiments in which the enzyme dose is about 730 μg to about 14.6 mg protein or a cellulase or cellulase cocktail characterized by an activity of about 2.67 to about 53.33 FPU.

Cellulases are preferably used at a dose range of about 10 CTU to about 500 CTU cellulase per gram dry weight of solids in the slurry (e.g., about 10 CTU, 20 CTU, 30 CTU, 40 CTU, 50 CTU, 60 CTIU, 80 CTU, 100 CTU, 125 CTU, 150 CTU, 175 CTU, 200 CTJU, 250 CTU, 300 CTU, 400 CTU or 500 CTU). In various embodiments, the amount of cellulase per gram dry weight of solids in the slurry is in a range bounded by any two of the foregoing embodiments, such as about 10 CTU to about 200 CTU, about 20 CTU to about 400 CTU, about 400 CTU to about 250 CTU, about 10 CTU to about 100 CTU, or about 20 CTU to about 250 CTU, etc.

### Primary Fermentation

The fermentation of the pentose sugars in the hydrolysate can be carried out by one or more appropriate fermenting microorganisms as set forth hereinabove in single or multistep fermentations.

In one embodiment, the fermentation of the pentose sugars in the hydrolysate using the pentose/hexose fermentation microorganism is accomplished in a fed-batch process. The fermentation vessels for the primary fermentation step include six vessels of about 300,000 gallon capacity. The fennentation vessels are agitated but are operated anaerobically without air supply.

The fermentation vessels will be charged with detoxified, neutralized hydrolysate and inoculated with yeast at about 1 g/L to about 2 g/L from yeast propagation. Primary fermentation will occur at pH of about 6.3, at a temperature of about 34° C. with a residence time of about 49 hours. During this time, most of the solubilized sugars will be fermented to ethanol, resulting in a primary fermentation broth of about 2 w/v % to about 4 w/v % ethanol. The primary fermentation broth after about 49 hours will also contain unfermented soluble pentose sugars, principally residual xylose, which will be further converted into ethanol in the secondary fermentation.

In another embodiment, the detoxified and/or neutralized hydrolysate stream will then be fed to a primary fermentor to which has been charged the propagated yeast and the nutrients. The initial volume of the inoculum and nutrient will be about 2% to about 5% of the working volume and the detoxified hydrolysate will be added for a period of time of at least about 7 hours to about 20 hours to make up the volume to ill working volume. During the fill time, the yeast will start converting the fermentable sugars to ethanol and carbon dioxide. The pH of this process will be maintained at about 5.5 to about 6.5 by the addition of ammonium hydroxide. The temperature will be maintained at about 32° C. to about 38° C. The exact pH and temperature optimum will be determined by the exact strain of yeast used and the composition/hydrolysis/detox conditions used in the upstream steps. The fill of the fermentation will continue until the desired working volume is met and the vessel will ferment the available sugars to point where the glucose, fructose and sucrose are completely utilized.

### Secondary Fermentation

The end product of enzymatic digestion of cellulose by the enzyme activities described hereinabove is the monomer sugar glucose. Because glucose inhibits betaglucosidase (β-G) activity, it acts as an inhibitor to the overall reaction. Therefore, many commonly used enzyme mixtures contain significant amounts of β-G activity to achieve high conversion yields. However, in the 1980s several researchers observed that this end product inhibition could be relieved by the addition of a fermenting organism, such as yeast, which consumes the glucose as it is produced by the enzyme system. Thus, the simultaneous saccharification and fermentation (SSF) of cellulose is a more efficient system, requiring less enzyme activity than a process that carries out separate saccharification hydrolysis followed by fermentation (SHF).

The secondary fermentation of the solids stream begins with using the broth product from primary fermentation to slurry the pretreated cake that results from solidiliquid separation. Pretreated cake, containing mostly cellulose and residual lignin, is fed into a slurry tank with the primary fermentation broth. Magnesium hydroxide is also added to the slurry tank to neutralize residual acid that comes with the pretreated cake. Optionally, cellulase enzymes from T. reesei enzyme can be added to the slurry tank in order to reduce the viscosity of the slurry. Up to about 16% of the total enzyme prep may be added to the slurry tank.

Optionally, the portion of primary fermentation broth used to sluny the solids can be heated treated prior to being fed into the slurry tank to ensure any contaminating microorganisms in the broth are inactivated or killed. In one embodiment, heat treatment is performed using an in-line heat exchanger elevating the temperature of the primary fermentation broth as it passes through to a temperature in the range of about 70° C. to about 100° C., and more preferably about 80° C. to about 85° C., for a period of time of about 1 second to about 60 seconds, and more preferably for about 45 seconds. Competing microorganisms if present in the primary fermentation broth will not be carry through to the secondary fermentation step.

Optionally, beta hops acids can be added to the secondary fermentation. Beta hops acids have a strong bacteriostatic effect against Gram positive bacteria and favor yeast such as S. cerevisiae. Again, competing microorganisms that might be present in the primary fermentation broth will be rendered inactive without having to subject the broth to a heat treatment step prior to secondary fermentation.

Fermentation vessels for the secondary fermentation include six 388,000 gallon fermenters. Inoculum for the secondary fermentation is carried with the primary fermentation broth into the slurry tank and eventually into the secondary fermenters. Secondary SSF will occur at a pH of about 5.0, about 35° C. with a residence time of about 30 hours. T. reesei enzyme preparation is added to the fermentation up to about 225 CTU/g solids. During this time sugars are simultaneously hydrolyzed from the solids cake and fermented by the yeast. The cake slurry is fed at solids concentration ranging about 14% to about 20% depending on the use of T. reesei preparation in the slurry tank for viscosity reduction. The secondary fermentation will result in a final broth with about 4% w/v to about 6% w/v ethanol.

In one embodiment, a volume of the primary fermentation broth is transferred to a secondary fermentor. A charge of the cellulolytic enzyme cocktail is added to the fermentor and the solids slurry is fed to the secondary fermentor over a period of about 5 hours to about 20 hours. During this time the solids slurry is pH adjusted by the addition of base to raise the pH above its average of about 1.5 to about 2.5 such that the addition of the low pH solids slurry does not lower the fermentation pH below the desired about 5.0 to about 5.5. Also the temperature of the solids slurry will be controlled such that the addition of the slurry does not take the secondary fermentor out of the range of about 32° C. to about 38° C. The enzyme cocktail will continue to be fed to the process at a rate of about 2% to about 3% final working volume. Over the course of the fill and subsequent fermentation time, the yeast will ferment all soluble sugars including glucose, fructose, xylose, and sucrose to ethanol and carbon dioxide. Concurrently, the enzymes will act on the cellulose in the solids and through the action of the enzymes will liberate glucose. This glucose will also be fermented to ethanol and carbon dioxide. This process will continue to either complete uptake and utilization of the fermentable sugars or the timing of the process dictates the secondary fermentation must be moved forward to free the fermentor for the following batch.

Optionally, to drive the reaction forward in the secondary fermentation step the pH can be variable. The optimal pH range for the saccharification enzymes is about 5.0 to about 5.5. This is lower than the optimal pH for the fermentation organism which is about 6 to about 7. By alternating the pH from a lower range to a higher range and back again the fermentation step can be optimized. At the optimal pH conditions for saccharification enzymes more glucose can be generated. At the optimal pH conditions for the fermentation organism the liberated glucose can be more readily converted to ethanol. The overall ethanol yield in the secondary fermentation step can be enhanced, and the time for the secondary fermentation step can potentially be decreased.

The fermentation broth is transferred to a large holding vessel, the broth well, where it is stored before being forwarded to the distillation unit. The ethanol is removed from the stream by distillation and will be upgraded through rectification and de-hydration to fuel grade ethanol. The process of distillation will also thermally inactivate the yeast ethanologen and the enzymes.

Stillage will be pumped to a centrifuge where the solids and liquids will be separated. The liquid fraction will be pumped to the waste treatment plant for digestion. The solids fraction will be diverted to a biomass boiler where the solids are burned as fuel to produce steam, or steam and electricity, which can be used in the process.

### Distillation and Dehydration

Fermentation products can be recovered using various methods known in the art. Products can be separated from other fermentation components by centrifugation, filtration, microfiltration, and nanofiltration. Products can be extracted by ion exchange, solvent extraction, or electrodialysis. Flocculating agents can be used to aid in product separation. As a specific example, bioproduced ethanol can be isolated from the fermentation medium using methods known in the art for ABE fermentations (see for example, Durre, 1998, Appl. Microbiol. Biotechnol. 49:639-648; Groot et al., 1992, Process. Biochem. 27:61-75; and references therein). For example, solids can be removed from the fermentation medium by centrifugation, filtration, decantation, or the like.

After fermentation, the fermentation product, e.g., ethanol can be separated from the fermentation broth by any of the many conventional techniques known to separate ethanol from aqueous solutions. These methods include evaporation, distillation, azeotropic distillation, solvent extraction, liquid-liquid extraction, membrane separation, membrane evaporation, adsorption, gas stripping, pervaporation, and the like.

### EXAMPLES

### Example 1

### Primary Fermentation

Primary fermentation was conducted as batch or fed-batch starting with about 15% of final volume and about 8 hour feed for the remaining volume. The fermentation media consisted of detoxified hydrolysate acting as carbon source mainly providing xylose but also comprising hexose sugars like sucrose, fructose and glucose. Hydrolysate and cellulosic solids (“cake”) were generated after a one step liquid solids separation step of dilute acid pretreated lignocellulosic feedstock. In addition to hydrolysate the fermentation broth contained KH₂PO₄ (3 mg/L final concentration) and MgSO₄.7H₂O. (0.5 mg/L final concentration) and pentose and hexose co-utilizing yeast at a final concentration of about 1 g/L (dry weight/volume). In the case of a fed-batch fermentation, the required amounts of MgSO₄.7H₂O and KH₂PO₄ were added to the initial start volume. Likewise all yeast was used to inoculate the starting volume of the fermentation. Supplies of about 28% ammonium hydroxide and about 42.5% phosphoric acid were used for all pH adjustments. Primary fermentations were conducted at pH of about 6.3 and about 32° C. and ran for about 43 hours under efficient mixing (about 200 rpm in 10 L Sartorius fermentation vessels). At the end of fermentation the broth was pasteurized by keeping the fermentation at about 55° C. for about 30 min.

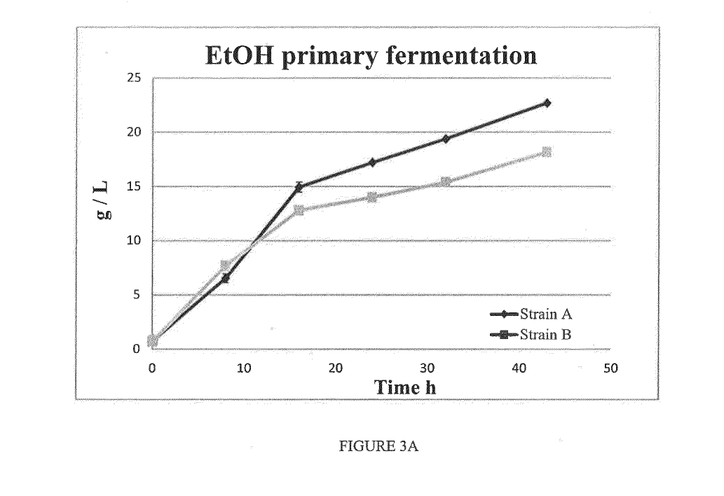

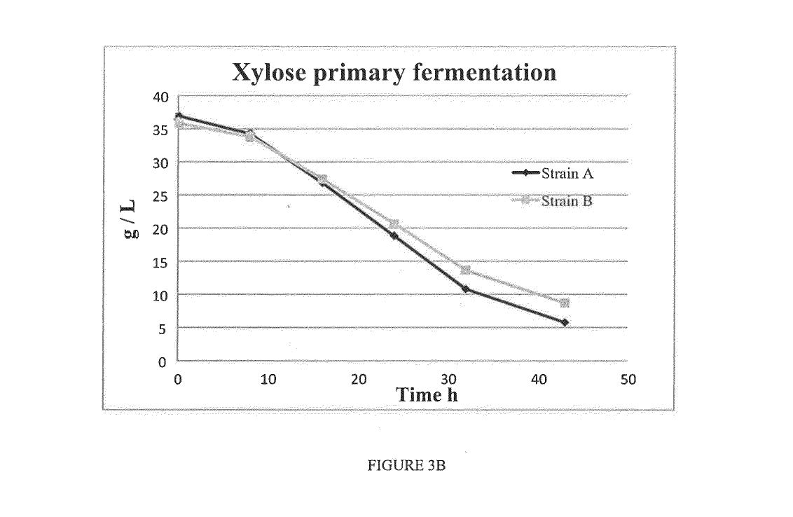

FIGS. 3A and 3B show ethanol production and xylose consumption in two separate runs using different S. cerevisiae yeast strains each capable of fermenting both pentose and hexose sugars.

### Example 2

### Secondary Fermentation

For the secondary fermentation the pasteurized primary fermentation broth was used to create slurry comprising primary fermentation broths, cellulosic solids (“cake”) and hydrolysate still entrained in the cake after the liquid solids separation step. The target consistency of the slurry was about 12% to about 13.5% of insoluble solids (w/v). Before fermentation the cake slurry was adjusted to a pH of about 5.5 using magnesium hydroxide. Secondary fermentations were conducted as fed-batch SSF fermentations starting with an initial volume of about 10% to about 15% of final volume. This initial volume included the complete dose of cellulosic enzyme required for the reaction (about 225 CTU T. reesei enzyme preparation+β-glucosidase) as well as the required yeast inoculum of pentose and hexose co-utilizing yeast at a final concentration of about 0.5 gDW/L (dry weight/volume). The remainder of the volume was made up by cake slurry fed in over about 8 hours. The secondary fermentation was conducted at about 35° C. and a pH of about 5.5 and efficient mixing (about 400 rpm in 10 L Sartorius fermentation vessels) for up to about 56 hours. Process pH was maintained using the same acid and base solutions as in the primary fermentation.

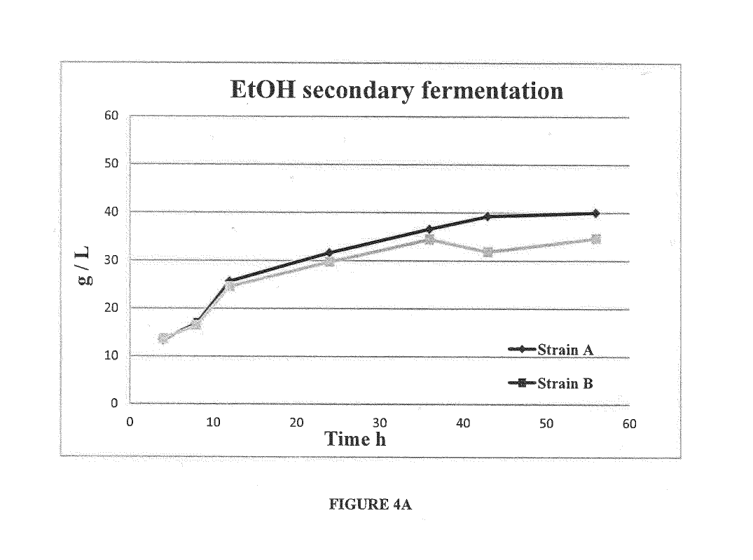

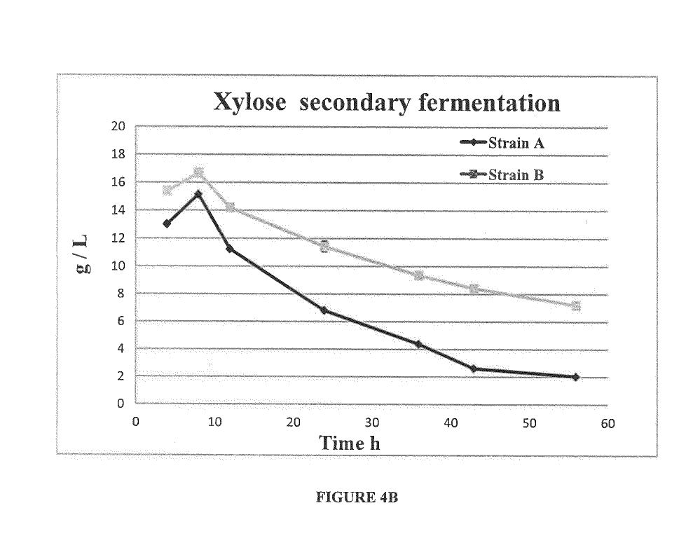

FIGS. 4A and 4B show ethanol production and xylose consumption in two separate runs using different S. cerevisiae yeast strains each capable of fermenting both pentose and hexose sugars in the secondary fermentation.

Numerous alterations of the invention disclosed herein will suggest themselves to those skilled in the art. However, it is to be understood that the present disclosure relates to various embodiments of the invention which is for purposes of illustration only and not to be construed as a limitation of the invention. All such modifications which do not depart from the spirit of the invention are intended to be included within the scope of the appended claims.

1. A method of producing renewable material comprising:
(a) converting biologically a hemicellulose-derived material to form a first mixture comprising a first renewable material; and
(b) converting a substantial amount of a material comprising cellulose and lignin in the presence of at least a portion of the first mixture to form a second mixture comprising a second renewable material.

2. The method of claim 1, wherein the step of converting in step (b) comprises saccharification of the material.

3. The method of claim 2, wherein the method further comprises fermentation of the second renewable material formed in step (b).

4. The method of claim 3, wherein fermentation and saccharification occur simultaneously.

5. The method of claim 1, wherein the step of converting in step (a) comprises fermentation.

6. The method of claim 3, wherein fermentation of step (a) is performed by a microorganism.

7. The method of claim 3, wherein the first and second renewable materials are the same.

8. The method of claim 3, wherein the first and second renewable materials are selected from the group consisting of alcohols and organic acids.

9. The method of claim 8, wherein the alcohol is selected from the group consisting of ethanol and butanol.

10. The method of claim 9, wherein the microorganism is the same.

11. The method of claim 3, wherein the hemicellulose-derived material and the material comprising cellulose and lignin are obtained from hydrolysis of a lignocellulosic feedstock.

12. The method of claim 11, wherein hydrolysis comprises the step of contacting the feedstock with an acid.

13. The method of claim 1, wherein the hemicellulose-derived material is detoxified prior to converting in step (a).

14. The method of claim 1, wherein the first mixture reduces the viscosity of the second mixture.

15. The method of claim 1, wherein step (a) and step (b) occur in the same vessel.

16. A method for producing an alcohol comprising:
(a) fermenting xylose in a mixture comprising a quantity of xylose during a first time to form a first mixture comprising an alcohol and unfermented xylose;
(b) saccharifying a material comprising cellulose and lignin to form glucose in a second mixture comprising at least a portion of the first mixture; and
(c) fermenting in the second mixture the glucose in the second mixture and at least a portion of the unfermented xylose from the first mixture, to form an alcohol.

17. The method of claim 16, wherein the fermenting in step (c) and the saccharifying occur simultaneously.

18. The method claim 16, wherein the fermenting in step (a) and the fermenting in step (b) are in separate vessels.

19. The method claim 16, wherein the fermenting in step (a) and the fermenting in step (b) are in the same vessel.

20. A method of producing renewable material comprising:
(a) converting biologically at least a portion of a hemicellulose-derived material to form a first mixture comprising a first renewable material; and
(b) converting biologically at least a portion of a cellulose-derived material in the presence of at least a portion of the first mixture and lignin to form a second mixture comprising a second renewable material.

21. A process for producing ethanol from lignocellulosic feedstock comprising:
(a) hydrolyzing in the presence of water the feedstock comprising lignin, cellulose and hemicellulose to form a liquid portion comprising xylose and a solids portion comprising cellulose and lignin;
(b) separating at least a substantial amount of the liquid portion from the solids portion to form a liquid fraction and a solids fraction;
(c) contacting the liquid fraction with a fermentation organism to form a first mixture comprising ethanol;
(d) mixing the solid fraction of step (b) with at least a portion of the first mixture generated in step (c) to form a slurry; and
(e) contacting the slurry of step (d) with an enzyme and the fermentation organism to form a second mixture comprising ethanol.
